(19) United States
(12) Patent Application Publication
(10) Pub. No.: US 2024/0091112 A1
(43) Pub. Date: Mar. 21, 2024

(54) Oral Compositions and Methods

(71) Applicant: Colgate-Palmolive Company, New York, NY (US)

(72) Inventors: Shaoyi ZHANG, Highland Park, NJ (US); Xiang CHEN, Somerset, NJ (US); Suman CHOPRA, Monroe, NJ (US); Cajetan DOGO-ISONAGIE, Mount Laurel, NJ (US)

(21) Appl. No.: 18/463,723

(22) Filed: Sep. 8, 2023

Related U.S. Application Data
(60) Provisional application No. 63/405,153, filed on Sep. 9, 2022.

(51) Int. Cl. A61K 8/22; A61K 8/23; A61K 8/24; A61K 8/25; A61K 8/34; A61K 8/36; A61K 8/42; A61K 8/46; A61K 8/81; A61K 8/86; A61Q 11/00

(52) U.S. Cl. CPC A61K 8/22; A61K 8/23; A61K 8/24; A61K 8/25; A61K 8/347; A61K 8/361; A61K 8/42; A61K 8/463; A61K 8/817; A61K 8/86; A61Q 11/00; A61K 2800/28; A61K 2800/48

(57) ABSTRACT

The present disclosure provides tooth whitening oral care compositions comprising potassium peroxymonosulfate and a source of hydrogen peroxide having improved stability, and methods of using the same.

### CROSS-REFERENCE TO RELATED APPLICATIONS

This application is an nonprovisional patent application which claims priority to, and the benefit of, U.S. Provisional Application Ser. No. 63/405,153, filed on Sep. 9, 2022, the contents of which are hereby incorporated by reference in its entirety.

### BACKGROUND

Consumer products providing for teeth whitening are numerous and take many forms, but one of the more popular forms are as dentifrices, such as toothpastes. Toothpastes must have a semisolid form, able to hold shape well enough to be dispensed from a tube and rest on toothbrush bristles, but also fluid enough to be easily squeezed from the tube. Toothpastes must also be sticky enough to adhere to some degree to the teeth, but also soluble enough to disperse in the oral cavity. These different aims are commonly satisfied by formulating toothpastes in a high-water base (e.g., 10-40% water) with a mixture of liquid polar humectants, such as glycerin, polypropylene glycol, and sorbitol. Often various polymers are used to provide the gel-like consistency that is necessary for a toothpaste.

Unfortunately, many whitening agents have stability problems in the presence of water, humectants, and some polymers. Other inorganic species, such as fluoride sources and surfactants, can also interact negatively, resulting in instability and loss of activity of the active whitening agent. It thus becomes necessary to formulate a whitening toothpaste with various ingredients intended to improve stability and activity of the whitening agent active.

Abrasives can be particularly difficult to formulate into whitening toothpastes, because of the high surface area, hygroscopicity, and acidity of many abrasives. Yet abrasives can be a critical component of a whitening composition because many stains adhere strongly but superficially to the tooth surface and an abrasive helps remove such stains both by its intrinsic abrasive action and by providing better access of the whitening agent to the stain.

Products that are presently available to whiten teeth include a variety of different ingredients, and the primary active whitening ingredient is most commonly a peroxide source, such as hydrogen peroxide. The use of peroxide agents often presents numerous difficulties in both formulation and long-term stability of the resulting compositions. In addition, in high concentrations, or in prolonged contact with the oral mucosa, hydrogen peroxide can be highly irritating to the teeth and gums. Thus, while hydrogen peroxide does have teeth whitening benefits, it can be difficult to utilize it in oral care products because of formulation, stability, and consumer acceptability issues.

Peroxysulfuric acid (H₂SO₅, also known as peroxymonosulfuric acid), and its salts, the peroxymonosulfates, are powerful oxidizing and stain removing agents. They are currently used for a variety of industrial and consumer purposes, including swimming pool treatment and denture cleaning. Peroxymonosulfate salts generally have the anion [HSO₅]⁻, in contrast to the related peroxydisulfate salts which have the anion [HS₂O₈]⁻. Peroxymonosulfate whitening products have been explored for some oral care applications, including whitening strips, mouthwashes, and toothpastes. One common peroxymonosulfate oxidizing agent is potassium peroxymonosulfate (KHSO₅), also referred to as potassium monoperoxysulfate and abbreviated as KMPS or MPS, and sold as part of the composition Oxone or Caroat.

The use of potassium peroxymonosulfate in oral care applications has been very limited by its instability in aqueous solution, especially in aqueous solution near or above neutral pH. Potassium peroxymonosulfate has been known to degrade even in the presence of small quantities of water and heat. Thus, potassium peroxymonosulfate whitening compositions face particular difficulties in formulation.

There remains a need for tooth whitening dentifrice products based on peroxymonosulfate and hydrogen peroxide that can overcome the stability, formulation, and consumer acceptability issues that can occur.

### BRIEF SUMMARY

The present disclosure relates to an oral care composition (e.g., toothpaste) combining potassium peroxymonosulfate (“MPS”) and hydrogen peroxide (“HP”) to deliver unexpectedly improved whitening efficacy relative to either MPS or HP alone. In one aspect, disclosed herein is at least one formulation strategy to stabilize the dual active agents (MPS and HP) which passes accelerated aging stability screening test. The disclosure also provides that the selected combination of MPS and HP (e.g., at 1% MPS and 2% HP) exhibits superior whitening efficacy compared to even a toothpaste having much higher levels of hydrogen peroxide (e.g., 5% w/v). Thus, in one aspect, the disclosure provides toothpaste formulations that can provide MPS combined with low amounts of hydrogen peroxide that provide improved whitening efficacy relative to formulations with high amounts of hydrogen peroxide alone, e.g., 5% hydrogen peroxide, and which avoids potential issues with stability and consumer acceptance.

The present disclosure provides a tooth whitening oral care composition comprising 0.01-10% potassium peroxymonosulfate (MPS) by weight of the composition, 5%-60% of an abrasive selected from calcium pyrophosphate (Ca₂P₂O₇), insoluble sodium metaphosphate ([NaPO₃]n), anhydrous dicalcium phosphate (CaHPO₄), or mixtures thereof by weight of the composition, and from 0.25%-25% by wt. of a source of hydrogen peroxide, by weight of the composition, wherein the source of hydrogen peroxide provides hydrogen peroxide (e.g., free hydrogen peroxide) in an amount from 0.05%-4.5% by wt. of the total composition. In some embodiments, the tooth whitening oral care composition comprises about 1% potassium peroxymonosulfate (MPS) and the source of hydrogen peroxide in an amount to provide about efficacy compared 3% hydrogen peroxide (e.g., free hydrogen peroxide) by wt. of the total composition. In further embodiments, the compositions may further comprise from 20-60% of a poloxamer (e.g., polyoxyethylene/polyoxypropylene triblock copolymer), by wt. of the composition. In further embodiments, the compositions may further comprise one or more of polyvinylpyrrolidone, sodium stearate, abrasive silica (e.g., up to 10% by weight), polyethylene glycol/polypropylene glycol random copolymer, polyethylene glycol, propylene glycol, alkali metal polyphosphates, anionic surfactants, and amphoteric surfactants. In at least one aspect, the tooth whitening oral care compositions of the present disclosure are low water or anhydrous compositions.

Further areas of applicability of the present disclosure will become apparent from the detailed description provided hereinafter. It should be understood that the detailed description and specific examples, while indicating the preferred embodiment of the invention, are intended for purposes of illustration only and are not intended to limit the scope of the invention.

### DETAILED DESCRIPTION

The following description of the preferred embodiment(s) is merely exemplary in nature and is in no way intended to limit the invention, its application, or uses.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

Open terms such as “include,” “including,” “contain,” “containing,” and the like, mean “comprising.” In this description, unless otherwise stated, the use of the singular also includes the plural. For example, “a lubricant” also comprehends the case where more than one lubricant is used.

“About” means plus or minus 20% of the stated value. Thus, for example, “about 5%” means from 80% to 120% of 5%, or 4.0% to 6.0%, inclusive of the end values of the range.

In a first aspect, the present disclosure provides a tooth whitening oral care composition (Composition 1) comprising (a) 0.01-10% potassium peroxymonosulfate by weight of the composition (e.g., about 1% by wt.), (b) 5%-60% of an abrasive selected from calcium pyrophosphate (Ca₂P₂O₇), insoluble sodium metaphosphate ([NaPO₃]ₙ), anhydrous dicalcium phosphate (CaHPO₄), or mixtures thereof, by weight of the composition, and (c) from 0.25%-25% of a source of hydrogen peroxide, by weight of the composition, wherein the source of hydrogen peroxide provides hydrogen peroxide (e.g., free hydrogen peroxide) in an amount from 0.05%-4.5% by wt. of the total composition (e.g., about 0.1%, about 1%, or about 2%, or about 3%, or about 4%, by wt.). In further embodiments, the present disclosure provides:
1.1. Composition 1, wherein the potassium peroxymonosulfate is provided as a triple salt of potassium peroxymonosulfate, potassium hydrogen sulfate and potassium sulfate, optionally wherein the triple salt comprises about 45%-50% by weight of potassium peroxymonosulfate, e.g., 47% or 49% by weight of potassium peroxymonosulfate;
1.2. Composition 1 or 1.1, wherein the composition comprises the potassium peroxymonosulfate in an amount of 0.01% to 5%, or 0.05% to 5%, or 0.1% to 5%, or 0.5% to 3%, or 0.5% to 2.5%, or 0.5% to 2%, or 0.5% to 1.5%, or 0.75% to 1.25%, or 1% to 5%, or 1% to 4%, or 1% to 3% or 1% to 2%, or 1.5% to 3%, or 2% to 3%, or 1.5% to 2%, or 2% to 2.5%, or about 0.5%, or about 1%, or about 1.5%, or about 2%, or about 2.5%, by weight of the composition;
1.3. Any of the preceding compositions, wherein the inorganic peroxymonosulfate salt is not particulated (e.g., not granulated);
1.4. Any of the preceding compositions, wherein the potassium peroxymonosulfate and the source of hydrogen peroxide are the only oxidizing agents present in the composition and/or the only whitening agents present in the composition;
1.5. Composition 1, or any of 1.1-1.4, wherein the composition comprises 5%-60% (e.g., 5-30%) calcium pyrophosphate (Ca₂P₂O₇), by weight of the composition;
1.6. Composition 1, or any of 1.1-1.4, wherein the composition comprises 5%-60% (e.g., 5-30%) insoluble sodium metaphosphate ([NaPO₃]ₙ), by weight of the composition;
1.7. Composition 1, or any of 1.1-1.4, wherein the composition comprises 5%-60% (e.g., 5-30%) calcium pyrophosphate (Ca₂P₂O₇) and insoluble sodium metaphosphate ([NaPO₃]ₙ), by weight of the composition;
1.8. Composition 1, or any of 1.1-1.4, wherein the composition comprises 5%-60% (e.g., 5-30%) anhydrous dicalcium phosphate (CaHPO₄), by weight of the composition;
1.9. Composition 1, or any of 1.1-1.4, wherein the composition comprises 5%-60% (e.g., 5-30%) calcium pyrophosphate (Ca₂P₂O₇) and anhydrous dicalcium phosphate (CaHPO₄), by weight of the composition;
1.10. Composition 1, or any of 1.1-1.9, wherein the composition comprises the calcium pyrophosphate and/or the insoluble sodium metaphosphate and/or the anhydrous dicalcium phosphate (CaHPO₄) in a total amount of 10% to 60%, or 10% to 50%, or 10% to 40%, or 10% to 35%, or 10% to 60%, or 10% to 50%, or 10% to 40%, or 10% to 35%, or 10% to 30%, or 10% to 30%, or 10% to 30%, or 10% to 27.5%, or 12% to 27%, or 12% to 26%, or about 15%, or about 20%, by weight of the composition;
1.11. Composition 1, or any of 1.1-1.10, wherein the composition comprises the calcium pyrophosphate and/or the insoluble sodium metaphosphate and/or the anhydrous dicalcium phosphate (CaHPO₄) each independently in an amount of 1% to 50%, or 1% to 40%, or 1% to 30%, or 1% to 25%, or 1% to 20%, or 1% to 10%, or 1% to 5%, or 10% to 35%, or 10% to 30%, or 10% to 20%, or 20% to 40%, or 20% to 30%, or 30% to 50%, or 30% to 40%, or 10% to 27.5%, or 15% to 25%, or 12% to 27%, or 12% to 26%, or about 10%, or about 15%, or about 20%, by weight of the composition;
1.12. Composition 1, or any of 1.1-1.10, wherein the source of hydrogen peroxide is selected from polymer-linked hydrogen peroxide complex, silica-hydrogen peroxide complex (e.g., 20-50% HP by weight, e.g., HP complexed with fumed silica), and liquid aqueous hydrogen peroxide (e.g., 35% aqueous hydrogen peroxide or 50% aqueous hydrogen peroxide);
1.13. Composition 1.13, wherein the source of hydrogen peroxide is a polymer-linked hydrogen peroxide complex;
1.14. Composition 1.13, wherein the source of hydrogen peroxide is polyvinylpyrrolidone-hydrogen peroxide complex;
1.15. Composition 1.13, wherein the source of hydrogen peroxide is cross-linked polyvinylpyrrolidone-hydrogen peroxide complex;
1.16. Composition 1.14 or 1.15, wherein the polyvinylpyrrolidone-hydrogen complex comprises 15% to 25% by weight of hydrogen peroxide, e.g., about 18% to 20% by weight of hydrogen peroxide, or about 18% by weight or about 20% by weight;
1.17. Composition 1, or any of 1.1-1.16, wherein the composition comprises from 0.5% to 25% of the source of hydrogen peroxide (e.g., cPVP-HP complex), by weight of the composition, e.g., 1% to 25%, or 1% to 20%, or 1% to 15%, or 1% to 10%, or 1% to 5%, or 5% to 25%, or 5% to 20%, or 5% to 15%, or 5% to 10%, or 10% to 25%, or 10% to 20%, or 10% to 15%, or 15% to 25%, or 15% to 20%, or 6% to 12%, 8% to 12%, or 16% to 17%, or about 5%, or about 10%, or about 15%, or about 16%, or about 17%, by weight of the composition;
1.18. Composition 1, or any of 1.1-1.17, wherein the source of hydrogen peroxide provides hydrogen peroxide (e.g., free hydrogen peroxide) in an amount from 0.05% to 4.5% by wt. of the total composition, e.g., 0.1% to 4.5%, or 0.5% to 4.5%, or 1 to 4.5%, or 1% to 4%, or 2% to 4%, or 3% to 4%, or 0.1% to 3.5%, or 0.5% to 3.5%, or 1% to 3.5%, or 2% to 3.5%. or 0.1% to 2.5%, or 0.5% to 2.5%, or 1% to 2.5%, or 1.5% to 2.5%, or 0.1% to 1%, or 0.1% to 0.5%, or about 0.1%, or about 1%, or about 2%, or about 3%, or about 4%, by weight of the composition;
1.19. Composition 1, or any of 1.1-1.18, wherein the composition further comprises a poloxamer (e.g., polyoxyethylene/polyoxypropylene triblock copolymer);
1.20. Composition 1.19, wherein the polyoxyethylene/polyoxypropylene triblock copolymer is a triblock copolymer having the formula

HO—[CH₂CH₂O]ₐ[—CH(CH₃)CH₂O-]_{b}[CH₂CH₂O]ₐ—H,
wherein a is an integer between 1 and 30, b is an integer between 10 and 60;
1.21. Composition 1.20, wherein in said formula, a is an integer between 5 and 20, and b is an integer between 10 and 40;
1.22. Composition 1.21, wherein in said formula, a is an integer between 10 and 15, and b is an integer between 10 and 20;
1.23. Composition 1.22, wherein in said formula, a is an integer between 10 and 12 (e.g., 11), and b is an integer between 15 and 20 (e.g., 16);
1.24. Composition 1, or any of 1.19-1.23, wherein the polyoxyethylene/polyoxypropylene triblock copolymer has an average molecular weight of 1000 to 7000 Daltons, e.g., 1000 to 6000 Daltons, or 1000 to 5000 Daltons, or 1000 to 4000 Daltons, or 1000 to 3000 Daltons, or 1000 to 2000 Daltons, or 1500 to 3000 Daltons, or 1500 to 2000 Daltons, or 1800 to 2000 Daltons, or about 1900 Daltons, optionally wherein said average molecular weight is a number average molecular weight or a weight average molecular weight;
1.25. Composition 1.24, wherein the polyoxyethylene/polyoxypropylene triblock copolymer is PLURONIC© L35 (i.e., poloxamer 407);
1.26. Composition 1, or any of 1.19-1.25, wherein the composition comprises the polyoxyethylene/polyoxypropylene triblock copolymer in an amount of 20% to 60%, e.g., 22% to 60%, or 22% to 50%, or 22% to 40%, or 22% to 35%, or 22% to 30%, or 25% to 60%, or 25% to 50%, or 25% to 40%, or 25% to 35%, or 25% to 30%, or 27% to 35%, or 28% to 35%, or about 26%, or about 30%, or about 31%, or about 32%, or about 33%, or about 34%, by weight of the composition;
1.27. Any preceding composition, wherein the composition further comprises one or more of polyvinylpyrrolidone (e.g., from 0.5 to 20% by wt.), polyethylene glycol/polypropylene glycol random copolymer (e.g., from 0.5%-50% by wt.), polyethylene glycol (e.g., from 0.5%-50% by wt.), humectants (e.g., polypropylene glycol, e.g., 1-20% by weight), polyphosphates (e.g., alkali metal polyphosphates, e.g., 0.1-5% by weight), surfactants (e.g., anionic and/or zwitterionic surfactants, e.g., 0.1-6% by weight), and thickening agents (e.g. fumed silica and/or sodium stearate, e.g., 0.1-10% by weight);
1.28. Composition 1.27, wherein the composition further comprises polyvinylpyrrolidone;
1.29. Composition 1.28, wherein the polyvinylpyrrolidone is cross-linked polyvinylpyrrolidone;
1.30. Composition 1.28 or 1.29, wherein the polyvinylpyrrolidone is not complexed with or combined with hydrogen peroxide (e.g., the polyvinylpyrrolidone is not added to the composition in the form of a PVP-hydrogen peroxide complex);
1.31. Composition 1.28 or 1.29, wherein the polyvinylpyrrolidone is complexed with or combined with hydrogen peroxide (e.g., cross-linked PVP complexed with hydrogen peroxide);
1.32. Composition 1.28, wherein the polyvinylpyrrolidone is linear polyvinylpyrrolidone;
1.33. Any of Compositions 1.28-1.32, wherein the composition comprises the polyvinylpyrrolidone in an amount of 1% to 50%, or 1% to 40%, or 1% to 30%, or 1% to 25%, or 1% to 22%, or 1% to 20%, or 1% to 18%, or 1% to 15%, or 1% to 12%, or 1% to 10%, or 1% to 8%, or 1% to 6%, or 3% to 15%, or 3% to 12%, or 3% to 10%, or 3% to 8%, or 3% to 6%, or 4% to 8%, or 4% to 6%, or 5% to 10%, or 6% to 8%, or about 5%, or about 7.5%, by weight of the composition;
1.34. Any of the preceding compositions, wherein the composition further comprises a polyethylene glycol/polypropylene glycol random copolymer (PEG/PPG copolymer, such as PEG/PPG 116/66 or PEG/PPG 38/8);
1.35. Composition 1.34, wherein the PEG/PPG random copolymer has an average molar ratio of ethylene glycol units (EG) to propylene glycol units (PG) of about 75-150 EG to 45-95 PG, or about 95-135 EG to 50 to 80 PG, or about 105-125 EG to 55-75 PG, or about 110-120 EG to 60-70 PG, or about 116 EG to 66 PG (i.e., PEG/PPG 116/66);
1.36. Composition 1.34 wherein the PEG/PPG random copolymer is PLURACARE® L1220 (PEG/PPG 116/66);
1.37. Any of Compositions 1.34-1.36, wherein the composition comprises the PEG/PPG random copolymer in an amount of 0.5% to 50%, or 1% to 50%, or 1% to 40%, or 1% to 30%, or 1% to 25%, or 1% to 20%, or 1% to 18%, or 1% to 15%, or 1% to 12%, or 1% to 10%, or 6% to 40%, or 6% to 30%, or 6% to 25%, or 6% to 20%, or 6% to 15%, or 6% to 10%, or 8% to 30%, or 8% to 25%, or 8% to 20%, or 8% to 15%, or 8% to 12%, or 10% to 30%, or 10% to 25%, or 10% to 20%, or 10% to 15%, or 10% to 12%, or about 10%, by weight of the composition;
1.38. Any of the preceding compositions, wherein the composition further comprises polyethylene glycol;
1.39. Composition 1.38, wherein the polyethylene glycol is selected from the group consisting of PEG-200, PEG-300, PEG-400, PEG-500, PEG-600, PEG-800, PEG-1000, PEG-1600, and PEG-2000;
1.40. Composition 1.39, wherein the polyethylene glycol is PEG 600;
1.41. Any of compositions 1.38-1.40, wherein the composition comprises the polyethylene glycol in an amount of 0.5% to 50%, or 1% to 50%, or 1% to 40%, or 1% to 30%, or 1% to 25%, or 1% to 20%, or 1% to 18%, or 1% to 15%, or 5% to 40%, or 5% to 30%, or 5% to 25%, or 5% to 20%, or 5% to 15%, or 8% to 40%, or 8% to 30%, or 8% to 25%, or 8% to 20%, or 8% to 15%, or 10% to 30%, or 10% to 25%, or 10% to 20%, or 10% to 15%, or 12% to 25%, or 12% to 20%, or 12% to 15%, or about 10%, or about 12% (e.g., about 12.5%), by weight of the composition;
1.42. Any of the preceding compositions, wherein the composition further comprises a humectant (e.g., glycerol, sorbitol, xylitol, propylene glycol, or a mixture thereof).
1.43. Composition 1.42, wherein the humectant comprises propylene glycol or consists of propylene glycol;
1.44. Composition 1.42 or 1.43, wherein the composition comprises the humectant (e.g., propylene glycol) in an amount of 1% to 20%, or 2% to 18%, or 5% to 15%, or 5% to 12%, or 5% to 10%, or about 7.5%, by weight of the composition;
1.45. Any preceding composition, wherein the composition further comprises one or more additional polymers, such as, any one or more of: polypropylene glycols, polysaccharides (e.g., cellulose derivatives, for example carboxymethyl cellulose, hydroxypropyl cellulose, hydroxypropyl methyl cellulose, ethyl cellulose, microcrystalline cellulose; or polysaccharide gums, for example xanthan gum, guar gum, or carrageenan gum, pectins, karaya gum); chitosans; dextrans; hyaluronic acid and sodium hyaluronates; synthetic anionic polymeric polycarboxylates, such as copolymers of maleic anhydride or acid with another polymerizable ethylenically unsaturated monomer, preferably methyl vinyl ether (e.g., copolymers in a 1:4 to 4:1 ratio of maleic anhydride/acid to methyl vinyl ether); polyphosphonic acids and polyphosphonates (i.e., polyphosphoesters); cross-linked carboxyvinyl copolymers; polyacrylic acid or polyacrylate polymers (e.g. carbomers); polyacrylamides, such as (2-hydroxypropyl)methacrylamide; other polyoxyethylene-polyoxypropylene copolymers (PEG-PPG) tri-block copolymers, such as poloxamer 105, 108, 122, 123, 124, 182, 183, 184, 185, 188, 212, 215, 217, 234, 235, 237, 238, 288, 333, 334, 335, 338, 402, 403, or 407); PEG-PPG tetrablock copolymers; other PEG/PPG random copolymers, such as PEG/PPG-38/8; polyamines; polyvinyl alcohols; polyoxazolines, such as poly(2-alkyl-2-oxazolines), e.g., methyl, ethyl, or isopropyl substituted polyoxazolines; and quaternary ammonium polymers;
1.46. Any of the preceding compositions, wherein the composition does not comprise any one or more of: polyacrylic acid or polyacrylate polymers (PAA), polyvinylpyrrolidone-vinyl acetate copolymers (PVP-VA), polyoxazoline polymers (PO), and mixtures thereof;
1.47. Any of the preceding compositions, wherein the composition comprises 30 to 70% by weight of polymers, e.g., 30 to 60% by weight, or 40 to 60% by weight, or 50 to 60% by weight, or 55 to 60% by weight, or 55 to 59% by weight, or 58 to 59% by weight;
1.48. Any of the preceding compositions, wherein the composition further comprises a polyphosphate or an organic cyclic polyphosphate, such as an alkali metal pyrophosphate, an alkali metal tripolyphosphate, an alkali metal tetraphosphate, an alkali metal hexametaphosphate, an alkali metal insoluble metaphosphate, an alkali metal phytic acid salt, or a mixture thereof;
1.49. Composition 1.48, wherein the composition comprises a sodium or potassium pyrophosphate, a sodium or potassium tripolyphosphate, a sodium or potassium tetraphosphate, a sodium or potassium phytic acid salt, or a mixture thereof;
1.50. Composition 1.49, wherein the composition comprises tetrasodium or tetrapotassium pyrophosphate;
1.51. Composition 1.49, wherein the composition comprises disodium pyrophosphate or dipotassium pyrophosphate;
1.52. Composition 1.49, wherein the composition comprises tetrasodium pyrophosphate and disodium pyrophosphate;
1.53. Any of composition 1.48-1.52, wherein the composition comprises from 0.1 to 5% by weight of polyphosphates, e.g., 0.5% to 5%, or 1% to 5%, or 2% to 5%, or 2.5% to 5%, or 3% to 5%, or 3.5% to 5%, or 4% to 5%, or 2.5% to 4.5%, or 3% to 4.5%, or 3.5% to 4.5%, or 2% to 4%, or 2.5% to 4%, or about 3%, or about 3.5%, or about 4% by weight of polyphosphates (e.g., about 3% tetrasodium pyrophosphate and about 1% disodium pyrophosphate);
1.54. Any preceding composition, wherein the composition further comprises one or more surfactants, e.g., anionic surfactants, cationic surfactants, non-ionic, and/or zwitterionic surfactants;
1.55. Composition 1.54, wherein the composition comprises a mixture of anionic and zwitterionic surfactants;
1.56. Composition 1.54 or 1.55, wherein said anionic surfactants are selected from: sodium lauryl sulfate, sodium laureth sulfate, sodium myreth sulfate, sodium lauroyl sarcosinate, sodium coconut monoglyceride sulfonates, sodium lauryl benzene sulfonate, sodium lauryl sulfoacetate, sodium N-methyl N-cocoyl taurate, sodium cocoyl isethionate, sodium dioctyl sulfosuccinate, and sodium cocomonoglyceride sulfate, and ammonium analogs thereof,
1.57. Composition 1.54 or 1.55, wherein said zwitterionic surfactants are selected from: cocamidopropyl betaine, cocamidopropyl sultaine, cocamidopropyl hydroxysultaine, lauramidopropyl betaine, lauramidopropyl sultaine, lauramidopropyl hydroxysultaine, oleamidopropyl betaine, oleamidopropyl sultaine, oleamidopropyl hydroxysultaine, tallowamideopropyl betaine, tallowamidopropyl sultaine, tallowamidopropyl hydroxysultaine, lauryl betaine, lauryl sultaine, lauryl hydroxysultaine, lauryldimethylamine oxide, and myristamine oxide;
1.58. Composition 1.54, wherein said cationic surfactants are selected from: cetylpyridinium chloride (CPC), cetrimonium bromide, benzalkonium chloride, benzethonium chloride (1-hexadecylcarbamoyl-ethyl)-trimethylammonium halide, (1-hexadecylcarbamoyl-2-phenyl-ethyl)-trimethylammonium halide, 1-hexadecylcarbamoyl-1,1-dimethyl-pyrrolidinium halide, and [2-(1H-indole-3-yl)-1-hexadecylcarbamoyl-ethyl)]-trimethylammonium halide, wherein said halide is optionally chloride, fluoride or bromide, or lauroyl arginine, ethyl lauroyl arginine ester hydrochloride, or disodium sebacoyl bis-lauramidolysine;
1.59. Composition 1.54, wherein said non-ionic surfactants are selected from: cocomonoethanolamide, cocodiethanolamide, laurylamidopropyl dimethylamine oxide, myristylamidopropyl dimethylamine oxide, and decyl glucoside;
1.60. Any of the preceding compositions, wherein the composition comprises sodium lauryl sulfate;
1.61. Any of the preceding compositions, wherein the composition comprises cocamidopropyl betaine;
1.62. Any of the preceding compositions, wherein the composition comprises a mixture of sodium lauryl sulfate and cocamidopropyl betaine;
1.63. Any of the preceding compositions, wherein the composition comprises 0.1 to 6% of surfactants, e.g., 0.1 to 5%, or 0.5% to 5%, or 1 to 5%, or 1.5% to 5%, or 2% to 5%, or 3% to 5%, or 4% to 5%, or 1% to 4%, or 2% to 4%, or 3% to 4%, or 2% to 5%, or 3% to 5%, or 1% to 3%, or 1% to 2%, or 2% to 3%, or 2% to 2.5%, or 1.5% to 2.5%, or 2.5% to 3%, or 2.25% to 2.75%, or 2.25% to 2.5%, or about 2%, or about 2.3%, by weight of the composition;
1.64. Any of the preceding compositions, wherein the composition comprises any one or more surfactants in an individual amount of 0.1% to 5% of surfactants, e.g., 0.1% to 4%, or 0.1% to 3%, or 0.1% to 2.5%, or 0.1% to 2%, or 0.1% to 1.5%, or 0.1% to 1%, or 0.1% to 0.5%, or 1% to 4%, or 2% to 4%, or 1% to 3%, or 2% to 3%, or 1.5% to 2.5%, or 2% to 2.5%, or about 0.3%, or about 2%, by weight of the composition;
1.65. Any of the preceding compositions, wherein the composition comprises 0.1% to 5%, or 1% to 5%, or 2% to 4%, or 1% to 3%, or 2% to 3%, or 1.5% to 2.5%, or 2% to 2.5%, or about 2%, of sodium lauryl sulfate, and/or 0.1% to 1%, or 0.1% to 0.5%, or about 0.3%, of cocamidopropyl betaine, by weight of the composition;
1.66. Any of the preceding compositions, wherein the composition comprises an anionic surfactant (e.g., sodium lauryl sulfate) and a zwitterionic surfactant (e.g., cocamidopropyl betaine) in a weight ratio of about 20:1 to 1:1, e.g., about 20:1 to 2:1, or about 15:1 to 3:1, or about 12:1 to 4:1, or about 10:1 to 5:1, or about 8:1 to 5:1, or about 7:1 to 5:1, or about 6:1;
1.67. Any preceding composition, wherein the composition further comprises an antioxidant, e.g., selected from butylated hydroxyanisole, butylated hydroxytoluene, vitamin A, carotenoids, vitamin E, flavonoids, polyphenols, ascorbic acid, and mixtures thereof,
1.68. The preceding composition, wherein the antioxidant is butylated hydroxyanisole (BHA) or butylated hydroxytoluene (BHT);
1.69. The preceding composition, wherein the antioxidant is butylated hydroxytoluene;
1.70. Any of the preceding compositions, wherein the composition comprises any one or more antioxidants in an individual amount of 0.001% to 1%, e.g., 0.01% to 0.5%, or 0.01% to 0.3%, or 0.01% to 0.1%, or 0.01% to 0.05%, or about 0.03%, by weight of the composition;
1.71. Any of the preceding compositions, wherein the composition further comprises one or more thickening agents, e.g., sodium stearate, magnesium aluminum silicate, or fumed silica, optionally in an amount of 0.1% to 10% by weight of the composition, e.g., 1% to 10%, or 2.5% to 10%, or 3% to 10%, or 2.5% to 7.5%, or 3% to 8%, or 3% to 6%, or 3% to 5%, or 3% to 4%, or about 3%, or about 3.5%, or about 4%, or about 5%, by weight of the composition;
1.72. Composition 1.71, wherein the thickening agent comprises sodium stearate;
1.73. Composition 1.71 or 1.72, wherein the thickening agent comprises fumed silica;
1.74. Any preceding composition, wherein the composition further comprises a fluoride source;
1.75. Composition 1.74, wherein the fluoride source is selected from sodium fluoride, sodium monofluorophosphate, and stannous fluoride, or mixtures thereof,
1.76. Composition 1.74 or 1.75, wherein the composition comprises 0.1% to 5% of fluoride source(s) by weight of the composition, e.g., 0.5% to 5%, or 0.5% to 3%, or 0.5% to 2%, or 0.5% to 1%, or about 1%, or about 0.75%, or about 0.45%, by weight of the composition;
1.77. Any preceding composition, wherein the composition further comprises an additional abrasive (i.e., in addition to the calcium pyrophosphate and/or the insoluble sodium metaphosphate and/or the anhydrous dicalcium phosphate);
1.78. Composition 1.77, wherein the additional abrasive is selected from abrasive silica (e.g., hydrated silica, precipitated silica), calcium carbonate, calcium orthophosphate, dicalcium orthophosphate, tricalcium phosphate, and arginine carbonate, e.g., in an amount of 0.1% to 10%, or 0.1% to 5%, or 1% to 5%, or 2.5% to 5%;
1.79. Any of the preceding compositions, wherein the composition further comprises abrasive silica (e.g., in an amount of up to 10% by wt. of the composition);
1.80. Composition 1.79, wherein the composition comprises the abrasive silica in an amount of 0.1% to 10%, or 1% to 8%, or 1% to 5%, or 1% to 3%, or 1% to 2%, or 2% to 8%, or 3% to 7%, or 5% to 10%, or 8% to 10%, by weight of the composition;
1.81. Any preceding composition, wherein the composition further comprises a desensitizing agent, e.g., in an amount from 0.1% to 5% by weight, such as potassium nitrate;
1.82. Any preceding composition, wherein the composition further comprises an enamel strengthening agent, e.g., in an amount from 0.1% to 5% by weight, such as zinc phosphate (e.g., about 1% by wt.);
1.83. Any preceding composition, wherein the composition further comprises one or more of flavors, sweeteners, and/or colors, e.g., in an amount of 0.1% to 5%, or 0.5% to 5%, or 1% to 5%, or 2% to 5%, or 2% to 3%;
1.84. Any preceding composition, wherein the composition is substantially anhydrous (e.g., less than 4%, or less than 3%, or less than 2%, or less than 1% water by weight of the composition);
1.85. Any preceding composition, wherein the composition has no added water;
1.86. Any preceding composition, wherein the composition does not comprise any acetate esters, for example, wherein the composition does not comprise any of: triacetin, glyceryl acetate, propylene glycol diacetate, ethylene glycol diacetate, and diethylene glycol diacetate;
1.87. Composition 1 or any of 1.1-1.86, wherein the composition comprises or consists of: potassium peroxymonosulfate (e.g., as MPS triple salt), cross-linked PVP-hydrogen peroxide complex, calcium pyrophosphate, PEG/PPG triblock copolymer (e.g., poloxamer 408), polyvinylpyrrolidone, PEG-PPG random copolymer (e.g., PEG/PPG-116/66), polyethylene glycol (e.g., PEG-600), polyphosphates (e.g., sodium pyrophosphates), anionic surfactant (e.g., sodium lauryl sulfate), zwitterionic surfactant (e.g., cocamidopropyl betaine), a fluoride source (e.g., sodium monofluorophosphate), a thickener (e.g., fumed silica or sodium stearate), and one or more sweeteners, flavors, and or colors, and optionally propylene glycol, and optionally an antioxidant (e.g., BHT);
1.88. Composition 1 or any of 1.1-1.86, wherein the composition comprises or consists of: potassium peroxymonosulfate (e.g., as MPS triple salt), cross-linked PVP-hydrogen peroxide complex, calcium pyrophosphate, PEG/PPG triblock copolymer (e.g., poloxamer 408), polyvinylpyrrolidone, PEG-PPG random copolymer (e.g., PEG/PPG-116/66), polyethylene glycol (e.g., PEG-600), polyphosphates (e.g., sodium pyrophosphates), anionic surfactant (e.g., sodium lauryl sulfate), a fluoride source (e.g., sodium monofluorophosphate), a thickener (e.g., fumed silica), and one or more sweeteners, flavors, and or colors, and optionally propylene glycol, and optionally an antioxidant (e.g., BHT);
1.89. Composition 1 or any of 1.1-1.86, wherein the composition comprises or consists of: potassium peroxymonosulfate (e.g., as MPS triple salt), cross-linked PVP-hydrogen peroxide complex, calcium pyrophosphate, PEG/PPG triblock copolymer (e.g., poloxamer 408), PEG-PPG random copolymer (e.g., PEG/PPG-116/66), polyethylene glycol (e.g., PEG-600), propylene glycol, polyphosphates (e.g., sodium pyrophosphates), anionic surfactant (e.g., sodium lauryl sulfate), a fluoride source (e.g., sodium monofluorophosphate), a thickener (e.g., fumed silica), and one or more sweeteners, flavors, and or colors, and optionally an antioxidant (e.g., BHT);
1.90. Any preceding composition, wherein the composition comprises from 20-60% by wt., of one or more poloxamers (e.g., polyoxyethylene/polyoxypropylene triblock copolymer), by weight of the composition;
1.91. The preceding composition, wherein the composition comprises from 20-60% polyoxyethylene/polyoxypropylene triblock copolymer, by wt. of the composition;
1.92. Any preceding composition, wherein the source of hydrogen peroxide is selected from the group consisting of: hydrogen peroxide, a cross-linked polyvinylpyrrolidone (PVP) hydrogen peroxide complex, a polyvinylpyrrolidone (PVP) hydrogen peroxide complex, and combinations thereof,
1.93. The preceding composition, wherein the source of hydrogen peroxide comprises cross-linked PVP complexed with hydrogen peroxide;
1.94. Any preceding composition, wherein the composition is a dentifrice, e.g., a toothpaste or a tooth gel;
1.95. Any preceding composition, wherein the composition has the consistency of a paste or gel (e.g., not a free-flowing liquid and not a solid, such as a solid powder or pellets);
1.96. Any of the preceding compositions, wherein the source of hydrogen peroxide, and/or the potassium peroxymonosulfate are, or may be, maintained in separate containers until the point of use (e.g., a dual-component composition);
1.97. Composition 1, or any of compositions 1.1-1.96, wherein the source of hydrogen peroxide, and/or the potassium peroxymonosulfate are, or may be, maintained in the same container up to the point of use (e.g., a single-component composition), optionally wherein the single component is a single-phase or a dual-phase component.
1.98. Any of the preceding compositions, wherein the source of hydrogen peroxide is present in an amount that provides hydrogen peroxide (e.g., free hydrogen peroxide) in an amount of 0.05 weight % to 4 weight %, based on a total weight of the oral care product or the whitening composition thereof (e.g., from 0.075 weight % to 3.5 weight %, or from 0.075 weight % to 3 weight %, or from 0.075 weight % to 2.5 weight %, or from 0.075 weight % to 2.25 weight %, or from 0.075 weight % to 2 weight %, or about 0.1%, or about 0.5%, or about 1%, or about 2%, or about 3%, or about 4%, or about 4.5%, all by weight of the total composition);
1.99. Any of the preceding compositions, wherein the composition comprises the potassium peroxymonosulfate in an amount of 0.01% to 5%, or 0.05% to 5%, or 0.1% to 5%, or 0.5% to 3%, or 0.5% to 2.5%, or 0.5% to 2%, or 0.5% to 1.5%, or 0.75% to 1.25%, or 1% to 5%, or 1% to 4%, or 1% to 3% or 1% to 2%, or 1.5% to 3%, or 2% to 3%, or 1.5% to 2%, or 2% to 2.5%, or about 0.5%, or about 1%, or about 1.5%, or about 2%, or about 2.5%, by weight of the composition, and wherein the composition also comprises the source of hydrogen peroxide in an amount that provides hydrogen peroxide (e.g., free hydrogen peroxide) in an amount of 0.05 weight % to 4 weight %, based on a total weight of the oral care product or the whitening composition thereof (e.g., from 0.075 weight % to 3.5 weight %, or from 0.075 weight % to 3 weight %, or from 0.075 weight % to 2.5 weight %, or from 0.075 weight % to 2.25 weight %, or from 0.075 weight % to 2 weight %, or about 0.1%, or about 0.5%, or about 1%, or about 2%, or about 3%, or about 4%, or about 4.5%, all by weight of the total composition);
1.100. Any of the preceding compositions, wherein the composition comprises the potassium peroxymonosulfate in an amount of 0.01% to 5%, by wt. of the total composition (e.g., from 0.1%-2.5%, or about 1%) and the source of hydrogen peroxide in an amount that provides hydrogen peroxide (e.g., free hydrogen peroxide) in an amount of 0.05 weight % to 4 weight %, (e.g., from 0.075-3 weight %, or from 0.075-2 weight %, or about 0.5%, or about 1%, or about 2%, or about 3%, or about 4%, or about 4.5%, all by weight of the total composition);
1.101. Any of the preceding compositions, wherein the composition comprises the potassium peroxymonosulfate in an amount of about 1%, by wt. of the total composition, and the source of hydrogen peroxide in an amount that provides hydrogen peroxide (e.g., free hydrogen peroxide) in an amount of about 2% or 3%, by wt. of the total composition;
1.102. Any of the preceding compositions, wherein the source of hydrogen peroxide comprises cross-linked PVP complexed with hydrogen peroxide in an amount of from 1%-25%, by wt., or from 1%-20% by wt., or from 2%-15% by wt., or from 3%-12% by wt., or about 5.5% by wt., or about 11% by wt., or about 16-17% by weight, and wherein the cross-linked PVP complexed with hydrogen peroxide provides hydrogen peroxide (e.g., free hydrogen peroxide) in an amount from 0.05-4.5% by wt., e.g., from 0.075%-3.5% by wt., or from 0.075%-3% by wt., or from 0.075%-2.5% by wt., or about 0.1% by wt., or about 1% by wt., or about 2% by wt., or about 3% by wt., or about 4% by wt.;
1.103. Any the preceding compositions, wherein the composition comprises: 0.01-10% potassium peroxymonosulfate by weight of the composition, 5%-60% calcium pyrophosphate (Ca₂P₂O₇) and/or insoluble sodium metaphosphate ([NaPO₃]ₙ) by weight of the composition, from 3%-20% of a source of hydrogen peroxide, by weight of the composition, by weight of the composition, wherein the source of hydrogen peroxide provides hydrogen peroxide (e.g., free hydrogen peroxide) in an amount from 0.5-3.5% by wt.); and from 20-60% by wt., of one or more poloxamers (e.g., polyoxyethylene/polyoxypropylene triblock copolymer), wherein the weight is relative to the total composition;
1.104. Any the preceding compositions, wherein the composition comprises: 0.01-10% potassium peroxymonosulfate by weight of the composition (e.g., about 1% by wt., or about 2% by wt.), 5%-60% calcium pyrophosphate (Ca₂P₂O₇) and/or insoluble sodium metaphosphate ([NaPO₃]ₙ) by weight of the composition (e.g., from 27%-35% by wt.), from 0.5%-25% of cross-linked PVP complexed with hydrogen peroxide, by weight of the composition, wherein the cross-linked PVP complexed with hydrogen peroxide provides hydrogen peroxide (e.g., free hydrogen peroxide) in an amount from 0.05-4.5% by wt. (e.g., about 0.1% by wt., or about 1% by wt., or about 2% by wt., or about 3% by wt., or about 4% by wt., or about 4.5% by wt.), and from 20-60% of a PEG/PPG triblock copolymer (e.g., PLURONIC© L35) by weight of the composition (e.g., from 27%-34%, by wt.);
1.105. Any of the preceding compositions, wherein the source of hydrogen peroxide, by weight of the composition, is in an amount from 0.25%-25% by wt. of the composition (e.g., from 0.5%-15% by wt., or 10-20% by wt., or 3%-7% by wt., or from 8%-12% by wt., or about 5.5% by wt., or about 11% by wt., or about 16-17% by wt.);
1.106. Any of the preceding compositions, wherein the source of hydrogen peroxide is selected from the group consisting: hydrogen peroxide, urea peroxide, sodium perborate, calcium peroxide, a cross-linked polyvinylpyrrolidone (PVP) hydrogen peroxide complex, a polyvinylpyrrolidone (PVP) hydrogen peroxide complex, sodium percarbonate, and combinations thereof,
1.107. Any of the preceding compositions, wherein the composition comprises or consists of:

| Ingredient | Weight % | |
|---|---|---|
| Potassium peroxymonosulfate | 0.1-5% | (e.g., about 1%) |
| Cross-linked Polyvinylpyrrolidone | 5%-20% | (e.g., about 11%) |
| complexed with hydrogen peroxide (e.g., | | |
| 18% hydrogen peroxide relative to the total | | |
| weight of the complex) | | |
| Calcium pyrophosphate | 10-20% | (e.g., about 15%) |
| PEG/PPG triblock copolymer (e.g., | 25-40% | (e.g., about 33%) |
| PLURONIC ® L35) | | |
| Polyvinylpyrrolidone | 1-15% | (e.g., about 5%) |
| PEG-PPG random copolymer (e.g., | 6-15% | (e.g., about 10%) |
| PEG/PPG-116/66) | | |
| Polyethylene glycol (e.g., PEG 600) | 5%-20% | (e.g., about 10%) |
| Propylene Glycol | 0-20% | (e.g., about 7.5%) |
| Polyphosphate (e.g., sodium pyrophosphates) | 2.5-5% | (e.g., about 3.5%) |
| Anionic Surfactant (e.g., sodium lauryl | 1-5% | (e.g., about 2%) |
| sulfate) | | |
| Zwitterionic Surfactant (e.g., | 0.1-1% | (e.g., about 0.3%) |

| | |
|---|---|
| cocamidopropyl betaine) | |
| Fluoride source (e.g., sodium | 0.1 to 2% (e.g., about |
| monofluorophosphate) | 0.76%) |
| Antioxidant (e.g., BHT) | 0 to 0.3% (e.g., about |
| | 0.03%) |
| Thickener (e.g., fumed silica or sodium | 2.5-5% (e.g., about 3% or |
| stearate) | 4%) |
| Sweeteners and flavors, and/or colors | 0.5-5% |
| Total | Q.S. (ca. 100) |

1.108. Any of the preceding compositions, wherein the composition comprises or consists of:

| Ingredient | Weight % | |
|---|---|---|
| Potassium peroxymonosulfate | 0.1-5% | (e.g., about 1%) |
| Cross-linked Polyvinylpyrrolidone | 1%-10% | (e.g., about 5.5%) |
| complexed with hydrogen peroxide (e.g., | | |
| 18% hydrogen peroxide relative to the total | | |
| weight of the complex) | | |
| Calcium pyrophosphate | 10-40% | (e.g., about 20%) |
| PEG/PPG triblock copolymer (e.g., | 25-40% | (e.g., about 30%) |
| PLURONIC ® L35) | | |
| Propylene Glycol | 0-20% | (e.g., about 7.5%) |
| Polyvinylpyrrolidone | 1-15% | (e.g., about 7.5%) |
| PEG-PPG random copolymer (e.g., | 6-15% | (e.g., about 10%) |
| PEG/PPG-116/66) | | |
| Polyethylene glycol (e.g., PEG 600) | 10-20% | (e.g., about 12%) |
| Polyphosphate (e.g., sodium pyrophosphates) | 2.5-5% | (e.g., about 4%) |
| Anionic Surfactant (e.g., sodium lauryl | 1-5% | (e.g., about 2%) |

| | |
|---|---|
| sulfate) | |
| Fluoride source (e.g., sodium | 0.1 to 2% (e.g., about |
| monofluorophosphate) | 0.76%) |
| Antioxidant (e.g., BHT) | 0 to 0.3% (e.g., about |
| | 0.04%) |
| Thickener (e.g., fumed silica) | 2.5-5% (e.g., about 3%) |
| Sweeteners and flavors, and/or colors | 0.5-5% |
| Total | ca. 100 |

1.109. Any of the preceding compositions, wherein the composition comprises or consists of:

| Ingredient | Weight % | |
|---|---|---|
| Potassium peroxymonosulfate | 0.1-5% | (e.g., about 1%) |
| Cross-linked Polyvinylpyrrolidone | 1%-10% | (e.g., about 5.5%) |
| complexed with hydrogen peroxide (e.g., | | |
| 18% hydrogen peroxide relative to the total | | |
| weight of the complex) | | |
| Calcium pyrophosphate | 5%-40% | (e.g., about 20%) |
| PEG/PPG triblock copolymer (e.g., | 25-40% | (e.g., about 30%) |
| PLURONIC ® L35) | | |
| Polyvinylpyrrolidone | 1-15% | (e.g., about 7.5%) |
| PEG-PPG random copolymer (e.g., | 6-15% | (e.g., about 10%) |
| PEG/PPG-116/66) | | |
| Polyethylene glycol (e.g., PEG 600) | 10-20% | (e.g., about 12%) |
| Propylene Glycol | 0-20% | (e.g., about 7.5%) |
| Polyphosphate (e.g., sodium pyrophosphates) | 2.5-5% | (e.g., about 4%) |
| Anionic Surfactant (e.g., sodium lauryl | 1-5% | (e.g., about 2%) |

| | |
|---|---|
| sulfate) | |
| Fluoride source (e.g., sodium | 0.1 to 2% (e.g., about |
| monofluorophosphate) | 0.76%) |
| Antioxidant (e.g., BHT) | 0 to 0.3% (e.g., about |
| | 0.04%) |
| Thickener (e.g., fumed silica) | 2.5-5% (e.g., about 3%) |
| Sweeteners and flavors, and/or colors | 0.5-5% |
| Total | Q.S. (ca. 100) |

1.110. Any of the preceding compositions, wherein the composition comprises or consists of:

| Ingredient | Weight % |
|---|---|
| Potassium peroxymonosulfate (e.g., as MPS | 0.1-5% (e.g., about 1%) |
| triple salt) | (e.g., 0.2-10% MPS triple |
| | salt, e.g., 2.2%) |
| Cross-linked Polyvinylpyrrolidone complexed | 1%-25% (e.g., about 16- |
| with hydrogen peroxide (e.g., 18% hydrogen | 17%) |
| peroxide relative to the total weight of the | |
| complex) | |
| Calcium pyrophosphate | 10-40% (e.g., about 15%) |
| PEG/PPG triblock copolymer (e.g., | 25-40% (e.g., about 26%) |
| PLURONIC ® L35) | |
| PEG-PPG random copolymer (e.g., | 6-15% (e.g., about 10%) |
| PEG/PPG-116/66) | |
| Polyethylene glycol (e.g., PEG 600) | 10-20% (e.g., about 10%) |
| Propylene glycol | 5-20% (e.g., about 7.5%) |
| Polyphosphate (e.g., sodium pyrophosphates) | 2.5-5% (e.g., about 3%) |
| Anionic Surfactant (e.g., sodium lauryl | 1-5% (e.g., about 2%) |
| sulfate) | |
| Fluoride source (e.g., sodium | 0.1 to 2% (e.g., about |
| monofluorophosphate) | 0.76%) |
| Antioxidant (e.g., BHT) | 0 to 0.3% (e.g., 0.03%) |
| Thickener (e.g., fumed silica) | 2.5-5% (e.g., about 3%) |
| Sweeteners and flavors, and/or colors | 0.5-5% (e.g., about 3%) |
| Total | ca. 100 |

1.111. Any preceding composition, wherein the composition comprises a total amount of water (including any comprised in another ingredient) of less than 4% by weight of the composition, e.g., less than 3% by weight, or less than 2% by weight, or less than 10% by weight, or less than 0.50% by weight, or less than 0.10%, or about 0.00010% to about 4% by weight, or about 0.000100 to about 0.50% by weight or about 0.000100 to about 0.10% by weight;
1.112. Any preceding composition, wherein the composition is a dentifrice, e.g., a toothpaste or tooth gel.

Potassium peroxymonosulfate (also known as MPS, KMPS, potassium monopersulfate, or potassium monoperoxysulfate) is commercially available as Caroat® or Oxone®, both of which are a triple salt of potassium peroxymonosulfate, potassium hydrogen sulfate and potassium sulfate (2KHSO₅·KHSO₄·K₂SO₄) (i.e., “MIPS triple salt”).

Potassium peroxymonosulfate has limited stability in aqueous solutions and can require other stabilizing ingredients. Therefore, contact with water during processing and storage should be avoided or minimized. The compositions are preferably packaged in a moisture free environment.

As used herein, the term “insoluble sodium metaphosphate” is used to refer to the insoluble polymeric sodium metaphosphate, which has the empirical formula [NaPO₃]ₙ, also known as “Maddrell's Salt.” This is a highly useful abrasive, which is insoluble in water and has a low capacity for releasing phosphate ion into solution. It has a high molecular weight, with values of n up to 2000. It is distinct from such soluble species as trisodium orthophosphate (Na₃PO₄), tetrasodium pyrophosphate (Na₄P₂O₇), pentasodium tripolyphosphate (Na₅P₃O₁₀), hexasodium tetraphosphate (Na₆P₄O₁₃), sodium trimetaphosphate (Na₃[(PO₃)₃)]), or sodium hexametaphosphate (Na₆[(PO₃)₆)]), all of which are water soluble and prone to hydrolysis under aqueous conditions to provide orthophosphate anion.

The compositions of the present disclosure contain no water or have a low water content. As used herein, the term “low water content” means the total concentration of water, including any free water and all water contained in any ingredients. In various embodiments of the composition, the amount of water is in an amount of less than 4% by weight, or less than 3% by weight, or less than 2% by weight, or less than 1% by weight, or less than 0.5% by weight, or less than 0.1%, or about 0.0001% to about 4% by weight, or about 0.0001% to about 0.5% by weight or about 0.0001% to about 0.1% by weight.

The amounts of potassium peroxymonosulfate and the source of hydrogen peroxide in the compositions of the invention is effective to result in improved tooth whitening when used once or twice daily for about three months as compared to a control composition without the peroxymonosulfate salt or hydrogen peroxide source. The amount of peroxymonosulfate salt typically is about 0.1% to about 10%, by weight of the composition, and the amount of the source of hydrogen peroxide is sufficient to provide about 0.5 to 5% hydrogen peroxide by weight of the composition.

In some embodiments, the compositions of the present disclosure contain a buffering agent. Examples of buffering agents include anhydrous carbonates such as sodium carbonate, sesquicarbonates, bicarbonates such as sodium bicarbonate, silicates, bisulfates, phosphates such as monopotassium phosphate and dipotassium phosphate, citrates, pyrophosphates (sodium and potassium salts) and combinations thereof. The amount of buffering agent is sufficient to provide a pH of about 5 to about 9, preferable about 6 to about 8, and more preferable about 7, when the strip is hydrated. Typical amounts of buffering agent are about 0.1% to about 5%, in one embodiment about 1% to about 3%, in another embodiment about 0.5% to about 1%, by weight of the total composition.

In certain embodiments, the compositions of the present disclosure (e.g., any of Composition 1, et seq.) comprise a poloxamer, which is a polyoxyethylene-polyoxypropylene triblock copolymer. The term “poloxamer” or “poloxamer copolymer” refers to a nonionic triblock copolymer composed of a central hydrophobic chain of polyoxypropylene units (a.k.a. poly(propylene oxide) units) flanked by two hydrophilic chains of polyoxyethylene units (e.g., poly(ethylene oxide) units). Poloxamers have the following chemical structure: HO—[CH₂CH₂O]ₐ[—CH(CH₃)CH₂O-]_{b}[CH₂CH₂O]ₐ—H, wherein a and b are integers, each typically between 10 and 200. Poloxamers are named according to common conventions based on their molecular weight and ethoxy content, and include poloxamer 407, poloxamer 338, poloxamer 237, poloxamer 188 and poloxamer 124. PLURONIC© is the name of a line of poloxamer polymers manufactured by BASF. For example, PLURONIC© F-127 is poloxamer 407. Poloxamers are distinguished from other polyethylene glycol/polypropylene glycol copolymers (PEG/PPG copolymers or EO/PO copolymers) which have a structure other than as a triblock structure, such as a random copolymer structure. Such copolymers that are distinct from poloxamers include the PEG/PPG copolymers sold by BASF as the PLURACARE® and PLURAFLOW® series polymers, which are random PEG/PPG copolymers.

For example, suitable poloxamers may include one or more of PLURONIC® L35, PLURONIC® L43, PLURONIC® L64, PLURONIC® L10, PLURONIC® L44, PLURONIC® L62, PLURONIC® 10R5, PLURONIC® 17R4, PLURONIC® L25R4, PLURONIC® P84, PLURONIC® P65, PLURONIC® PI 04, and PLURONIC® PI 05. PLURONIC® brand dispersants are commercially available from BASF, Florham Park, NJ.

In some embodiments, the compositions of the present disclosure may comprise polyvinylpyrrolidone (optionally cross-linked), also known as poly-N-vinyl-poly-2-pyrrolidone, and commonly abbreviated to cross-linked “PVP.” PVP generally refers to a polymer containing vinylpyrrolidone (also referred to as N-vinylpyrrolidone, N-vinyl-2-pyrrolidione and N-vinyl-2-pyrrolidinone) as a monomeric unit. The monomeric unit may include a polar imide group, four non-polar methylene groups, and a non-polar methane group. Cross-linked PVP includes those commercially available as KOLLIDON® and LUVICROSS®, marketed by BASF, Mount Olive, N.J., USA; and POLYPLASDONE® INF-10, marketed by, Ashland, Covington, Kentucky, USA.

The source of hydrogen peroxide may be or include any compound or material capable of or configured to provide or release hydrogen peroxide. In one aspect, the source of hydrogen peroxide may be configured to release hydrogen peroxide when contacted with water. In certain aspects, illustrative sources of hydrogen peroxide may be or include, but are not limited to, hydrogen peroxide (aqueous hydrogen peroxide), urea peroxide, sodium perborate, calcium peroxide, a cross-linked polyvinylpyrrolidone (PVP) hydrogen peroxide complex, a polyvinylpyrrolidone (PVP) hydrogen peroxide complex, a silica-hydrogen peroxide complex, sodium percarbonate, and combinations thereof. The sources of hydrogen peroxide may also be or include, but are not limited to, PEROXYDONE® XL 10F complex, which is commercially available from Ashland Inc. of Covington, Ky. In a typical implementation, the source of hydrogen peroxide is aqueous hydrogen peroxide or cross-linked PVP-hydrogen peroxide complex.

The amount or concentration of the source of hydrogen peroxide may depend upon the amount of hydrogen peroxide provided or otherwise delivered by the source of hydrogen peroxide. In some embodiments, the source of hydrogen peroxide may be present in an amount (e.g., from 0.25%-25% by wt.) to provide from 0.5%-4.5%, by weight, of hydrogen peroxide (e.g., free hydrogen peroxide), based on a total weight of the oral care composition. For example, the source of hydrogen peroxide may be present in an amount (e.g., from 0.5-20% by wt., or about 5.5% by wt., or about 11% by wt., or about 16-17% by wt.) that provides hydrogen peroxide (e.g., free hydrogen peroxide) in an amount of 0.05 weight % to 4 weight %, based on a total weight of the oral care product or the whitening composition thereof. In some embodiments, the oral care composition of the disclosure (e.g., any of Composition 1, et seq) comprises a source of hydrogen peroxide in an amount (e.g., from 0.5-20% by wt., or about 5.5% by wt., or about 11% by wt., or about 16-17% by wt.) that provides hydrogen peroxide (e.g., free hydrogen peroxide) from 0.075 weight % to 3 weight %, based on the total weight of the oral care composition. In some embodiments, the oral care composition of the disclosure (e.g., any of Composition 1, et seq) comprises a source of hydrogen peroxide in an amount (e.g., from 0.5-15% by wt. or about 5.5% by wt., or about 11% by wt.) that provides hydrogen peroxide (e.g., free hydrogen peroxide) in an amount from 0.075 weight % to 2.5 weight %, based on the total weight of the oral care composition. In some embodiments, the oral care composition of the disclosure (e.g., any of Composition 1, et seq) comprises a source of hydrogen peroxide in an amount (e.g., from 0.5-15% by wt. or about 5.5% by wt., or about 11% by wt.) that provides hydrogen peroxide (e.g., free hydrogen peroxide) in amount from of 0.075 weight % to 2.25 weight %, based on a total weight of the oral care composition. In some embodiments, the oral care composition of the disclosure (e.g., any of Composition 1, et seq) comprises a source of hydrogen peroxide in an amount (e.g., from 0.5-15% by wt., or about 5.5% by wt., or about 11% by wt.) that provides hydrogen peroxide (e.g., free hydrogen peroxide) in an amount from of 0.075 weight % to 2 weight %, based on a total weight of the oral care composition. In some embodiments, the oral care composition of the disclosure (e.g., any of Composition 1, et seq) comprises a source of hydrogen peroxide in an amount (e.g., from 0.5-25% by wt., or about 5.5% by wt., or about 11% by wt., or about 16-17% by wt., or about 20-22% by wt., or about 25% by wt.) that provides about 0.05%, or about 0.1%, or about 1%, or about 2%, or about 3%, or about 4%, or about 4.5%, hydrogen peroxide by wt., wherein the weight is based on a total weight of the oral care composition.

In some embodiments, the compositions of the present disclosure (e.g., any of Composition 1, et seq.) can optionally contain whitening (oxidizing) agents in addition to the potassium peroxymonosulfate and the source of hydrogen peroxide. Whitening agents are generally materials which are effective to provide whitening of a tooth surface to which it is applied, and include agents such as urea peroxide. In various embodiments, the compositions of the present disclosure (e.g., any of Composition 1, et seq.) comprise a source of hydrogen peroxide, wherein the source of hydrogen peroxide comprises a bivalent oxygen-oxygen group. In various embodiments, the compositions of the present disclosure (e.g., any of Composition 1, et seq) may further comprise peroxides of alkali and alkaline earth metals, organic peroxy compounds, peroxy acids, pharmaceutically-acceptable salts thereof, and mixtures thereof. Peroxides of alkali and alkaline earth metals include lithium peroxide, potassium peroxide, sodium peroxide, magnesium peroxide, calcium peroxide, barium peroxide, and mixtures thereof. Organic peroxy compounds include carbamide peroxide (also known as urea hydrogen peroxide), glyceryl hydrogen peroxide, alkyl hydrogen peroxides, dialkyl peroxides, alkyl peroxy acids, peroxy esters, diacyl peroxides, benzoyl peroxide, and monoperoxyphthalate, and mixtures thereof. Peroxy acids and their salts include organic peroxy acids such as alkyl peroxy acids, and monoperoxyphthalate and mixtures thereof, as well as inorganic peroxy acid salts such as persulfate, dipersulfate, percarbonate, perphosphate, perborate and persilicate salts of alkali and alkaline earth metals such as lithium, potassium, sodium, magnesium, calcium and barium, and mixtures thereof. In certain embodiments, the oral care compositions of the disclosure (e.g., any of Composition 1, et seq) further comprises urea peroxide and/or sodium percarbonate, in addition to the source of hydrogen peroxide. In some embodiments, the compositions of the disclosure (e.g., any of Composition 1 et seq) may further comprise a non-peroxide whitening agent. Whitening agents among those useful herein include non-peroxy compounds, such as chlorine dioxide, chlorites and hypochlorites. Chlorites and hypochlorites include those of alkali and alkaline earth metals such as lithium, potassium, sodium, magnesium, calcium and barium. Non-peroxide whitening agents also include colorants, such as titanium dioxide and hydroxyapatite. One or more additional whitening agents are optionally present in a tooth-whitening effective total amount. In some embodiments the compositions additionally comprise an activator, e.g., tetraacetylethylenediamine. In some embodiments, the composition of the present invention may comprise non-oxidizing whitening agents, which are agents which achieve a whitening effect without oxidizing stains in the teeth, such as, titanium dioxide or blue dye or color (e.g., FD&C Blue color, or blue 15 pigment (CI 74160)). In some embodiments, the compositions of the present invention are free of all of the above enumerated additional whitening agents.

The compositions of the present disclosure optionally can also include other ingredients, e.g., flavor agents; fillers; surfactants; preservatives, e.g., sodium benzoate and potassium sorbate; color agents including, e.g., dyes and pigments; and sweeteners. In some embodiments, the compositions of the present disclosure comprise one or more surfactants, such as anionic, cationic, zwitterionic or non-ionic surfactants.

As used herein, “anionic surfactant” means those surface-active or detergent compounds that contain an organic hydrophobic group containing generally 8 to 26 carbon atoms or generally 10 to 18 carbon atoms in their molecular structure and at least one water-solubilizing group selected from sulfonate, sulfate, and carboxylate so as to form a water-soluble detergent. Usually, the hydrophobic group will comprise a C₈-C₂₂ alkyl, or acyl group. Such surfactants are employed in the form of water-soluble salts and the salt-forming cation usually is selected from sodium, potassium, ammonium, magnesium and mono-, di- or tri-C₂-C₃ alkanolammonium, with the sodium, magnesium and ammonium cations again being the usual ones chosen. Some examples of suitable anionic surfactants include, but are not limited to, the sodium, potassium, ammonium, and ethanolammonium salts of linear C₈-C₁₈ alkyl ether sulfates, ether sulfates, and salts thereof. Suitable anionic ether sulfates have the formula R(OC₂H₄)ₙOSO₃M wherein n is 1 to 12, or 1 to 5, and R is an alkyl, alkylaryl, acyl, or alkenyl group having 8 to 18 carbon atoms, for example, an alkyl group of C₁₂-C₁₄ or C₁₂-C₁₆, and M is a solubilizing cation selected from sodium, potassium, ammonium, magnesium and mono-, di- and triethanol ammonium ions. Exemplary alkyl ether sulfates contain 12 to 15 carbon atoms in the alkyl groups thereof, e.g., sodium laureth (2 EO) sulfate. Some preferred exemplary anionic surfactants that may be used in the compositions of the present disclosure include sodium laurel ether sulfate (SLES), sodium lauryl sulfate, and ammonium lauryl sulfate. In certain embodiments, the anionic surfactant is present in an amount of 0.01 to 5.0%, 0.1 to 2.0%, 0.2 to 0.4%, or about 0.33%.

As used herein, “nonionic surfactant” generally refers to compounds produced by the condensation of alkylene oxide groups (hydrophilic in nature) with an organic hydrophobic compound which may be aliphatic or alkyl-aromatic in nature. Examples of suitable nonionic surfactants include poloxamers (sold under trade name PLURONIC®), polyoxyethylene, polyoxyethylene sorbitan esters (sold under trade name TWEENS®), Polyoxyl 40 hydrogenated castor oil, fatty alcohol ethoxylates, polyethylene oxide condensates of alkyl phenols, products derived from the condensation of ethylene oxide with the reaction product of propylene oxide and ethylene diamine, ethylene oxide condensates of aliphatic alcohols, alkyl polyglycosides (for example, fatty alcohol ethers of polyglycosides, such as fatty alcohol ethers of polyglucosides, e.g., decyl, lauryl, capryl, caprylyl, myristyl, stearyl and other ethers of glucose and polyglucoside polymers, including mixed ethers such as capryl/caprylyl (C₈₋₁₀) glucoside, coco (C₈₋₁₆) glucoside, and lauryl (C₁₂₋₁₆) glucoside), long chain tertiary amine oxides, long chain tertiary phosphine oxides, long chain dialkyl sulfoxides, and mixtures of such materials.

In some embodiments, the nonionic surfactant comprises amine oxides, fatty acid amides, ethoxylated fatty alcohols, block copolymers of polyethylene glycol and polypropylene glycol, glycerol alkyl esters, polyoxyethylene glycol octylphenol ethers, sorbitan alkyl esters, polyoxyethylene glycol sorbitan alkyl esters, and mixtures thereof. Examples of amine oxides include, but are not limited to, laurylamidopropyl dimethylamine oxide, myristylamidopropyl dimethylamine oxide, and mixtures thereof. Examples of fatty acid amides include, but are not limited to, cocomonoethanolamide, lauramide monoethanolamide, cocodiethanolamide, and mixtures thereof. In certain embodiments, the nonionic surfactant is a combination of an amine oxide and a fatty acid amide. In certain embodiments, the amine oxide is a mixture of laurylamidopropyl dimethylamine oxide and myristylamidopropyl dimethylamine oxide. In certain embodiments, the nonionic surfactant is a combination of lauryl/myristylamidopropyl dimethylamine oxide and cocomonoethanolamide. In certain embodiments, the nonionic surfactant is present in an amount of 0.01 to 5.0%, 0.1 to 2.0%, 0.1 to 0.6%, 0.2 to 0.4%, about 0.2%, or about 0.5%.

As used herein, the term “cationic surfactant” includes the cationic surfactants disclosed in WO 2007/011552A2, the contents of which are incorporated herein by reference in its entirety. Examples of such compounds include benzalkonium chloride, dodecyl trimethyl ammonium chloride, benzyl dimethyl stearyl ammonium chloride, hexadecyltrimethyl ammonium bromide, benzethonium chloride (diisobutyl phenoxyethoxyethyl dimethyl benzyl ammonium chloride), methyl benzethonium chloride, hexadecylpyridinium chloride, alkyl isoquinolinium bromides, hexetidine, hexamidine isethionate, octenadine, octenidine dihydrochioride, mono-biguanides (such as p-chlorobenzyl-biguanide and N′-(4-chlorobenzyl)-N″-(2,4-dichlorobenzyl) biguanide), poly(biguanides) (such as polyhexamethylene biguanide hydrochloride), bis-biguanides, cetyl pyridinium chloride, chlorhexidine, alexidine, and N-alpha-acyl arginine alkyl ester salts (such as ethyl lauroyl arginine ester hydrochloride (ELAH)).

As used herein, the term “zwitterionic surfactant” are generally linear compounds having both anionic and cationic groups in the polar part of the molecule, for example, carboxylic acid or sulfonic acid groups, and quaternary amine or amino oxide groups, respectively. Suitable zwitterionic surfactants include the betaines and the sultaines. Betaines are characterized by having a quaternary alpha-aminomethylcarboxylic acid group, while sultaines are characterized by having a quaternary gamma-aminopropylsulfonic acid group or gamma-amino-beta-hydroxysulfonic acid group. Suitable zwitterionic surfactants include, for example, cocamidopropyl betaine, cocamidopropyl sultaine, cocamidopropyl hydroxysultaine, lauramidopropyl betaine, lauramidopropyl sultaine, lauramidopropyl hydroxysultaine, oleamidopropyl betaine, oleamidopropyl sultaine, oleamidopropyl hydroxysultaine, tallowamideopropyl betaine, tallowamidopropyl sultaine, tallowamidopropyl hydroxysultaine, lauryl betaine, lauryl sultaine, lauryl hydroxysultaine, lauryldimethylamine oxide, and myristamine oxide.

Examples of the surfactant that can be used for compositions of the present disclosure include sodium lauryl sulfate, sorbitan fatty acid ester, polyoxyethylene (20) sorbitan monooleate (Polysorbate 80 or Tween 80), polyethylene glycol fatty acid ester, polyoxyethylene sorbitan fatty acid ester, polyoxyethylene alkyl ether, polyoxyethylene polyoxypropylene alkyl ether, polyoxyethylene polyoxypropylene block copolymer, polyoxyethylene alkyl phenyl ether, polyoxyethylene castor oil, polyoxyethylene hydrogenated castor oil, polyoxyethylene sorbitol fatty acid ester and polyoxyethylene glycerol fatty acid ester, and cocamidopropyl betaine. In the present invention, each of them may be used solely or two or more thereof may be used jointly. Typical amounts of surfactant are about 0.1% to about 3%, in one embodiment about 0.1% to about 2%, in another embodiment about 0.1% to about 1%, by weight of the total composition.

Examples of the filler are crystalline cellulose, ethylcellulose, dextrin, various kinds of cyclodextrin (alpha-cyclodextrin, beta-cyclodextrin, and gamma-cyclodextrin), sodium sulfate, as well as derivatives thereof and pullulan.

Useful flavor agents include natural and synthetic flavoring sources including, e.g., volatile oils, synthetic flavor oils, flavoring aromatics, oils, liquids, oleoresins and extracts derived from plants, leaves, flowers, fruits, stems and combinations thereof. Suitable flavor agents include, e.g., citric oils, e.g., lemon, orange, grape, lime and grapefruit, fruit essences including, e.g., apple, pear, peach, grape, strawberry, raspberry, cherry, plum, pineapple, apricot, and other fruit flavors. Other useful flavor agents include, e.g., aldehydes and esters (e.g., benzaldehyde (cherry, almond)), citral, i.e., alpha-citral (lemon, lime), neral, i.e., beta-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanal (green fruit), 2-dodedenal (citrus, mandarin) and mixtures thereof.

Suitable coloring agents include, e.g., food, drug and cosmetic (FD&C) colors including, e.g., dyes, lakes, and certain natural and derived colorants. Useful lakes include dyes absorbed on aluminum hydroxide and other suitable carriers.

Suitable sweetening agents include stevia, sugars such as sucrose, glucose, invert sugar, fructose, ribose, tagalose, sucralose, maltitol, erythritol, xylitol, and mixtures thereof, saccharin and its various salts (e.g., sodium and calcium salt of saccharin), cyclamic acid and its various salts, dipeptide sweeteners (e.g., aspartame), acesulfame potassium, dihydrochalcone, glycyrrhizin, and sugar alcohols including, e.g., sorbitol, sorbitol syrup, mannitol and xylitol, and combinations thereof.

It is understood that while general attributes of each of the above categories of materials may differ, there may be some common attributes and any given material may serve multiple purposes within two or more of such categories of materials. All of the ingredients in the compositions may have functions in addition to their primary function, and may contribute to the overall properties of the composition, including its stability, efficacy, consistency, mouthfeel, taste, odor and so forth. For example, a binder may also function as a disintegrating agent and vice versa.

In a second aspect, the present disclosure provides a method for whitening teeth comprising the steps of (a) applying Composition 1, or any of 1.1 et seq., to the teeth, and (b) maintaining contact of the composition with the teeth for a sufficient period of time (e.g., 0.1 to 60 minutes, or 0.1 to 30 minutes, or 0.1 to 10 minutes, or 0.1 to 5 minutes, or 0.1 to 2 minutes, or 0.1 to 1 minute) to effect whitening of the teeth contacted by the composition. In some embodiments, the composition may be applied using a toothbrush, and the composition maintained in contact with the teeth by using a brushing action. In some embodiments, the composition may be applied to the teeth using a dental tray, and the composition maintained in contact with the teeth by placement of the dental tray in the mouth until whitening is complete.

In other embodiments, the present disclosure provides for the use Composition 1, or any of 1.1 et seq., or any other embodiments thereof, for the whitening of the teeth.

### EXAMPLES

Exemplary embodiments of the present disclosure will be illustrated by reference to the following examples, which are included to exemplify, but not to limit the scope of the present invention.

In the examples and elsewhere in the description of the invention, chemical symbols and terminology have their usual and customary meanings. Temperatures are in degrees Celsius unless otherwise indicated. The amounts of the components are in weight percent based on the standard described; if no other standard is described then the total weight of the composition is to be inferred. Various names of chemical components include those listed in the CTFA International Cosmetic Ingredient Dictionary (Cosmetics, Toiletry and Fragrance Association, Inc., 7^{th} ed. 1997).

### Example 1: Exemplary HP/NIPS-Based Whitening Dentifrice

Potassium peroxymonosulfate is combined with calcium pyrophosphate and cross-linked PVP-hydrogen peroxide complex, and other excipients, and mixed to provide a homogenous product.

The compositions can have a formula as follows:

General Composition A

| Ingredient | Weight % | |
|---|---|---|
| Potassium peroxymonosulfate | 0.1-5% (e.g., about 1%) | |
| Cross-linked | 5%-20% (e.g., about | |
| Polyvinylpyrrolidone complexed | 11%) | |
| with hydrogen peroxide (e.g., | | |
| 18% hydrogen peroxide relative | | |
| to the total weight of the | | |
| complex) | | |
| Calcium pyrophosphate | 10-20% | (e.g., about 15%) |
| PEG/PPG triblock copolymer | 25-40% | (e.g., about 33%) |
| (e.g., PLURONIC ® L35) | | |
| Polyvinylpyrrolidone | 1-15% | (e.g., about 5%) |
| PEG-PPG random copolymer | 6-15% | (e.g., about 10%) |
| (e.g., PEG/PPG-116/66) | | |
| Polyethylene glycol (e.g., PEG | 10-20% | (e.g., about 10%) |
| 600) | | |
| Propylene Glycol | 0-20% | (e.g., about 7.5%) |
| Polyphosphate (e.g., sodium | 2.5-5% | (e.g., about 3.5%) |
| pyrophosphates) | | |
| Anionic Surfactant (e.g., sodium | 1-5% | (e.g., 2%) |
| lauryl sulfate) | | |
| Zwitterionic Surfactant (e.g., | 0.1-1% | (e.g., 0.3%) |
| cocamidopropyl betaine) | | |
| Fluoride source (e.g., sodium | 0.1 to 2% | (e.g., 0.76%) |
| monofluorophosphate) | | |
| Antioxidant (e.g., BHT) | 0 to 0.3% | (e.g., 0.03%) |
| Thickener (e.g., fumed silica) | 2.5-5% | (e.g., 4%) |

| | |
|---|---|
| Sweeteners and flavors, and/or | 0.5-5% |
| colors | |
| Total | ca. 100 |

General Composition B

| Ingredient | Weight % | |
|---|---|---|
| Potassium peroxymonosulfate | 0.1-5% (e.g., about 1%) | |
| Cross-linked | 1%-10% (e.g., about | |
| Polyvinylpyrrolidone complexed | 5.5%) | |
| with hydrogen peroxide (e.g., | | |
| 18% hydrogen peroxide relative | | |
| to the total weight of the | | |
| complex) | | |
| Calcium pyrophosphate | 10-40% | (e.g., about 20%) |
| PEG/PPG triblock copolymer | 25-40% | (e.g., about 30%) |
| (e.g., PLURONIC ® L35) | | |
| Propylene Glycol | 0-20% | (e.g., about 7.5%) |
| Polyvinylpyrrolidone | 1-15% | (e.g., about 7.5%) |
| PEG-PPG random copolymer | 6-15% | (e.g., about 10%) |
| (e.g., PEG/PPG-116/66) | | |
| Polyethylene glycol (e.g., PEG | 10-20% | (e.g., about 12%) |
| 600) | | |
| Polyphosphate (e.g., sodium | 2.5-5% | (e.g., about 4%) |
| pyrophosphates) | | |
| Anionic Surfactant (e.g., sodium | 1-5% | (e.g., about 2%) |

| | |
|---|---|
| lauryl sulfate) | |
| Fluoride source (e.g., sodium | 0.1 to 2% (e.g., about |
| monofluorophosphate) | 0.76%) |
| Antioxidant (e.g., BHT) | 0 to 0.3% (e.g., about |
| | 0.04%) |
| Thickener (e.g., fumed silica) | 2.5-5% (e.g., about 3%) |
| Sweeteners and flavors, and/or | 0.5-5% |
| colors | |
| Total | ca. 100 |

For example, an MPS/HP dentifrice composition (e.g., 1% MPS/3% HP) may be formulated according to the present disclosure:

| Ingredient | Weight % |
|---|---|
| Potassium peroxymonosulfate | 0.1-5% (e.g., about 1%) |
| (e.g., as MPS triple salt) | (e.g., 0.2-10% MPS triple |
| | salt, e.g., 2.2%) |
| Cross-linked | 1%-25% (e.g., about |
| Polyvinylpyrrolidone complexed | 16%) |
| with hydrogen peroxide (e.g., | |
| 18% hydrogen peroxide relative | |
| to the total weight of the | |
| complex) | |
| Calcium pyrophosphate | 10-40% (e.g., about 15%) |
| PEG/PPG triblock copolymer | 25-40% (e.g., about 26%) |
| (e.g., PLURONIC ® L35) | |
| PEG-PPG random copolymer | 6-15% (e.g., 10%) |
| (e.g., PEG/PPG-116/66) | |
| Polyethylene glycol (e.g., PEG | 10-20% (e.g., 10%) |
| 600) | |
| Propylene glycol | 5-20% (e.g., about 7.5%) |
| Polyphosphate (e.g., sodium | 2.5-5% (e.g., about 3%) |
| pyrophosphates) | |
| Anionic Surfactant (e.g., sodium | 1-5% (e.g., about 2%) |
| lauryl sulfate) | |
| Fluoride source (e.g., sodium | 0.1 to 2% (e.g., about |
| monofluorophosphate) | 0.76%) |
| Antioxidant (e.g., BHT) | 0 to 0.3% (e.g., about |
| | 0.03%) |
| Thickener (e.g., fumed silica) | 2.5-5% (e.g., about 3%) |
| Sweeteners and flavors, and/or | 0.5-5% (e.g., about 3%) |
| colors | |
| Total | ca. 100 |

Testing of the formulas within the scope of the disclosure demonstrates that they provide improved stability and retained active oxygen activity compared to comparative formulas not within the scope of the present disclosure.

### Example 2a: Formulation of MPS and HP Toothpaste

The combination of potassium peroxymonosulfate (“MPS”) and hydrogen peroxide in a toothpaste formulation is developed in order to utilize the dual active agents to unexpectedly increase the whitening efficacy of relative to toothpaste containing higher amounts of hydrogen peroxide.

First, the level of MPS is selected to be 1%, by wt. of the tested oral care composition. Then the level of HP is varied from 2% by wt., 3% by wt., or 4% by wt., to combine with MPS in order to evaluate the whitening efficacy. One challenge is developing a formulation strategy to deliver a stable dual active agent product. Without being bound by theory, challenges involved in developing a formulation strategy may relate to the understanding that formulating MPS or HP in a toothpaste alone presents one of skill in the art with limited ingredient choices due to poor compatibility. For example, commonly used humectants in certain market-based peroxide containing toothpastes and non-whitening toothpastes can include propylene glycol, glycerin, and sorbitol. However, some of these humectants cannot be formulated in MPS and HP containing toothpastes due to incompatibility with MPS. Consequently, the humectants for use in formulating General Compositions A and B (as described in Example 1) were PLURONIC© L35 (poloxamer 407), PEG (PEG-600), and propylene glycol (0-20%).

Second, a series of peroxide sources are also evaluated in an accelerated aging study, including cross-linked PVP-hydrogen peroxide complex and silica-hydrogen peroxide complex. A compatibility test of MPS with HP is designed by dispensing 1% MPS (provided as 2% by weight of Caroat triple salt), and 2% HP (either PVP-HP complex or silica-HP complex in an amount to provide 2% hydrogen peroxide by weight) into a gel base composed of 95% Pluronic L35 (poloxamer 407) and 5% sodium stearate. The samples are subjected to accelerated aging for 1 week at 60° C. and active oxygen content is measured initially and at 1 week. As expected, the compatibility of PVP-HP is improved relative to composition containing silica HP when combined with MPS. As shown in Table 1, it is found that the initial active oxygen content of the two compositions is similar, but after 1 week of aging, significantly higher active oxygen is retained in the composition made using PVP-HP complex, compared to that made using silica-HP complex.

TABLE 1
Stability of 1% MPS + 2% HP
with different HP sources in simple solutions.

| Active Oxygen | 1% MPS + 2% PVP HP | 1% MPS + 2% Silica HP |
|---|---|---|
| Initial | 2.30% | 2.38% |
| 60° C., 1 week | 1.88% | 1.22% |

Furthermore, the thickening agents fumed silica, cross-linked PVP, and sodium stearate are evaluated in full toothpaste formulas for viscosity and stability. The test formulas are as follows:

| Ingredient | Weight % | Weight % |
|---|---|---|
| Potassium peroxymonosulfate | 1% (2.2% MPS | 1% (2.2% MPS |
| | triple salt) | triple salt) |
| cPVP-HP complex (18 wt. % | 11% (providing 2% | 11% (providing 2% |
| hydrogen peroxide) | hydrogen peroxide) | hydrogen peroxide) |
| Calcium pyrophosphate | 15% | |
| Poloxamer 407 | 27% | 31% |
| Polypropylene glycol | 7.5% | 7.5% |
| PEG/PPG-116/66 random | 10% | 10% |
| copolymer | | |
| Polyethylene glycol (PEG 600) | 10% | 10% |
| Sodium pyrophosphates | 3.4% | 3.4% |
| Sodium lauryl sulfate | 2% | 2% |
| Sodium monofluorophosphate | 0.76% | 0.76% |
| Antioxidant (BHT) | 0.03% | 0.03% |
| Fumed silica (thickener) | 3.3% | 3.0% |
| Sodium stearate (thickener) | 0% | 0.4% |
| Cross-linked | 4.7% | 0% |
| polyvinylpyrrolidone | | |
| (thickener) | | |
| Sweeteners and Flavors | 3% | 3% |
| Total | Q.S. | Q.S. |

The samples are subjected to accelerated aging for 13 weeks at 40° C. and viscosity, pH, and active oxygen content are measured initially and at 13 weeks. It is found that each thickening agent is able to provide sufficient thickening efficiency and compatibility with 1% NIPS (provided by 2% Caroat) and 2% HIP (11% cPVP-HP complex). In addition, the combinations of fumed silica plus cross-linked PVP, or fumed silica plus sodium stearate, are also able to provide sufficient thickening efficiency and compatibility with MPS and HP. The results for the combination tests are shown in Table 2 below. As shown below, both thickening systems provide stable pH and good stability, with only a small and comparable decrease in active oxygen after 13 weeks of aging. Both thickening systems also provide acceptable viscosity, with a slightly higher viscosity provided by the fumed silica plus cPVP system.

TABLE 2
Viscosity and stability of 1% MPS + 2% HP toothpastes
with different thickening systems

| | | Fumed silica and | Fumed silica and |
|---|---|---|---|
| Thickening system: | | cross-linked PVP | sodium stearate |
| Initial | Viscosity | 210,861 cP | 185,607 cP |
| | pH | 7.5 | 8.2 |
| | Active Oxygen | 2.27% | 2.25% |
| 40° C., | pH | 7.3 | 7.8 |
| 13 weeks | Active Oxygen | 2.04% | 2.02% |

### Example 2b: Formulation Stability and Enamel Whitening Efficacy Relative to Hydrogen Peroxide Only Toothpaste

To evaluate stability, variations of Formula A, shown below, with 1% MPS and amounts of HP ranging from 2 to 4% are submitted to an accelerated aging study for 13 weeks at 40° C. Viscosity, pH, and active oxygen content are measured initially and at 13 weeks. The compositions of Formula A are shown in the following table:

### Formula A

| Ingredient | Weight % |
|---|---|
| Potassium peroxymonosulfate | 1% (2.2% MPS triple salt) |
| cPVP-HP complex (18 wt. % | 11%, 16.5%, or 22% |
| hydrogen peroxide) | (2%, 3%, or 4% hydrogen |
| | peroxide) |
| Calcium pyrophosphate | 15% |
| Poloxamer 407 | 33% |
| Polyvinylpyrrolidone (cPVP) | 5% |
| PEG/PPG-116/66 random | 10% |
| copolymer | |
| Polyethylene glycol (PEG 600) | 10% |
| Sodium pyrophosphates | 3.5% |
| Sodium lauryl sulfate | 2% |
| Cocamidopropyl betaine | 0.3% |
| Sodium monofluorophosphate | 0.76% |
| Antioxidant (BHT) | 0.03% |
| Fumed silica thickener | 4% |
| Sweeteners and Flavors | 0.5-5% |
| Total | Q.S. |

All data collected from the study was in specification, and no bloating or phase separation was observed throughout the aging, indicating that the combinations of 100 MPS and 24% HP all maintain good stability throughout aging. The results are shown in Table 3.

TABLE 3
Stability of toothpastes with 1% MPS and 2-4% HP

| | | 1% MPS 2% HP | 1% MPS + 3% HP | 1% MPS + 4% HP |
|---|---|---|---|---|
| | | Toothpaste | Toothpaste | Toothpaste |
| specification | pH | 7-8 | | |
| specification | AO | 1.99%-2.43%; | 2.89%-3.53%; | 379%-4.63%; |
| (initial; aged) | | >1.55% | >2.25% | >2.95% |
| Initial | pH | 7.92 | 7.9 | 7.84 |
| | Active Oxygen | 2.2% | 3.09% | 4.11% |
| 40 C. 13 | pH | 7.87 | 7.74 | 7.62 |
| weeks | Active Oxygen | 2.19% | 2.97% | 4.06% |

Formula A with 2% hydrogen peroxide is tested against a market-based 500 hydrogen peroxide whitening toothpaste. The test uses a standard in vitro brushing protocol that incorporates stained bovine teeth. The composition of the 500 HIP toothpaste is shown as follows:

| Ingredient | Weight % |
|---|---|
| Cross-linked PVP complexed with Hydrogen Peroxide | 22 |
| Calcium pyrophosphate | 15 |
| Polyethylene Glycol/Polypropylene Glycol random | 7.5 |
| copolymer | |
| 116/66Copolymer | |
| Polyoxypropylene-Polyoxyethylene triblock Copolymer | 45 |
| Fumed Silica | 3.0 |
| Polyphosphate | 1.7 |
| Butylated Hydroxytoluene | 0.03 |
| Sodium monofluorophosphate | 0.76 |
| Sodium lauryl sulfate | 2 |
| Sweeteners and Flavors | 2.98 |

The bovine central incisors are artificially stained (in coffee and tea broth) and mounted in a resin. The teeth are pre-brushed to have a starting L* value between 58 and 62. 10 g of the toothpastes are dispersed in 10 g of DI Water and the slurries are used to brush the bovine incisors for 2 minutes under a 250 g force at 120 strokes/min. L*, a*, b* values are measured using hyperspectral camera (Middleton Spectral Vision). The L* a*, b* values are measured after every two brushings, and 14 brushings are conducted to evaluate the whitening performance of 7 days usage of the toothpaste. The whitening performance is reported as the change in W value after treatments compared to baseline value, results are demonstrated in Tables 1 and 2 below. The equations used to calculate ΔW from L*, a*, b* values are shown below. In Table 1 and Table 2, the absolute ΔW is used to have positive values. The higher the absolute ΔW value, the whiter the tooth compared to the baseline measurement.

W*=(a*²+b*²+(L*−100)²)^{1/2}

ΔW=W*_{treated}−W*_{baseline}

The results of the test are shown in Table 4 below. The results demonstrate that composition according to the present invention, having 1% MPS and 2% HP, provides superior whitening compared to a commercial toothpaste product having 5% HP.

TABLE 4
Absolute (ΔW) as determined by Spectra shade values

| | | Formula A Toothpaste |
|---|---|---|
| Time Point | 5% HP Toothpaste | (1% MPS + 2% HP) |
| Baseline | 0 | 0 |
| Day 1 | 5.02 | 9.42 |
| Day 2 | 8.34 | 12.56 |
| Day 3 | 10.84 | 13.78 |
| Day 4 | 12.10 | 14.22 |
| Day 5 | 12.70 | 14.91 |
| Day 6 | 13.54 | 15.14 |
| Day 7 | 13.82 | 15.28 |

The invention has been described above with reference to illustrative Examples, but it is to be understood that the invention is not limited to the disclosed embodiments. Alterations and modifications that would occur to one of skill in the art upon reading the specification are also within the scope of the invention, which is defined in the appended claims.

1. A tooth whitening oral care composition comprising (a) 0.01-10% potassium peroxymonosulfate by weight of the composition, 5%-60% of an abrasive selected from calcium pyrophosphate (Ca₂P₂O₇), insoluble sodium metaphosphate ([NaPO₃]ₙ), anhydrous dicalcium phosphate (CaHPO₄), or mixtures thereof, by weight of the composition, and (c) from 0.25-25% of a source of hydrogen peroxide, by weight of the composition, wherein the source of hydrogen peroxide provides hydrogen peroxide (e.g., free hydrogen peroxide) in an amount from 0.05%-4.5% by wt. of the total composition (e.g., about 0.1%, about 1%, or about 2%, or about 3%, or about 4%, by wt.).

2. The composition of claim 1, wherein the composition comprises the potassium peroxymonosulfate in an amount of 0.01% to 5%, or 0.05% to 5%, or 0.1% to 5%, or 0.5% to 3%, or 0.5% to 2.5%, or 0.5% to 2%, or 0.5% to 1.5%, or 0.75% to 1.25%, or 1% to 5%, or 1% to 4%, or 1% to 3% or 1% to 2%, or 1.5% to 3%, or 2% to 3%, or 1.5% to 2%, or 2% to 2.5%, or about 0.5%, or about 1%, or about 1.5%, or about 2%, or about 2.5%, by weight of the composition.

3. The composition of claim 1, wherein the potassium peroxymonosulfate and the source of hydrogen peroxide are the only oxidizing agents present in the composition and/or the only whitening agents present in the composition.

4. The composition of claim 1, wherein the composition comprises 5-60% calcium pyrophosphate (Ca₂P₂O₇) by weight of the composition.

5. The composition of claim 1, wherein the source of hydrogen peroxide is selected from polymer-linked hydrogen peroxide complex, silica-hydrogen peroxide complex (e.g., 20-50% HP by weight, e.g., HP complexed with fumed silica), and liquid aqueous hydrogen peroxide (e.g., 35% aqueous hydrogen peroxide or 50% aqueous hydrogen peroxide).

6. The composition of claim 5, wherein the source of hydrogen peroxide is cross-linked polyvinylpyrrolidone-hydrogen peroxide complex.

7. The composition of claim 1, wherein the composition comprises a polyoxyethylene/polyoxypropylene triblock copolymer, and wherein the triblock copolymer has the formula
HO—[CH₂CH₂O]ₐ[—CH(CH₃)CH₂O—]_{b}[CH₂CH₂O]ₐ—H,
wherein a is an integer between 1 and 30, b is an integer between 10 and 60.

8. The composition of claim 1, wherein the composition further comprises one or more of polyvinylpyrrolidone (e.g., from 1%-50% by wt.), polyethylene glycol (e.g., from 0.5%-50% by wt.), humectants (e.g., polypropylene glycol, e.g., 1-20% by weight), polyphosphates (e.g., alkali metal polyphosphates, e.g., 0.1-5% by weight), surfactants (e.g., anionic and/or zwitterionic surfactants, e.g., 0.1-6% by weight), and thickening agents (e.g. fumed silica and/or sodium stearate, e.g., 0.1-10% by weight).

9. The composition of claim 8, wherein the composition comprises sodium lauryl sulfate and/or cocamidopropyl betaine.

10. The composition of claim 9, wherein the composition comprises 2 to 4%, or 1 to 3%, or 2 to 3%, or 1.5 to 2.5%, or 2 to 2.5%, or about 2%, of sodium lauryl sulfate, and/or 0.1 to 1%, or 0.1 to 0.5%, or about 0.3%, of cocamidopropyl betaine, by weight of the composition.

11. The composition of claim 1, wherein the composition further comprises a polyethylene glycol/polypropylene glycol random copolymer (PEG/PPG copolymer, such as PEG/PPG 116/66 or PEG/PPG 38/8), for example, in an amount of 0.5% to 50%, by weight of the composition.

12. The composition of claim 1, wherein the composition further comprises a humectant (e.g., glycerol, sorbitol, xylitol, propylene glycol, or a mixture thereof), optionally wherein the humectant comprises propylene glycol in an amount of 1 to 20% by weight of the composition.

13. The composition of claim 1, wherein the composition further comprises one or more thickening agents, e.g., sodium stearate, magnesium aluminum silicate, or fumed silica, optionally in an amount of 0.1% to 10% by weight of the composition, e.g., 1% to 10%, or 2.5% to 10%, or 3% to 10%, or 2.5% to 7.5%, or 3% to 8%, or 3% to 6%, or 3% to 5%, or 3% to 4%, or about 3%, or about 3.5%, or about 4%, or about 5%, by weight of the composition.

14. The composition of claim 13, wherein the composition comprises cross-linked polyvinylpyrrolidone and fumed silica, or sodium stearate and fumed silica.

15. The composition according to claim 1, wherein the composition comprises or consists of: (A) potassium peroxymonosulfate (e.g., as MPS triple salt), cross-linked PVP-hydrogen peroxide complex, calcium pyrophosphate, PEG/PPG triblock copolymer (e.g., poloxamer 408), polyvinylpyrrolidone, PEG-PPG random copolymer (e.g., PEG/PPG-116/66), polyethylene glycol (e.g., PEG-600), polyphosphates (e.g., sodium pyrophosphates), anionic surfactant (e.g., sodium lauryl sulfate), zwitterionic surfactant (e.g., cocamidopropyl betaine), a fluoride source (e.g., sodium monofluorophosphate), a thickener (e.g., fumed silica or sodium stearate), and one or more sweeteners, flavors, and or colors, and optionally propylene glycol, and optionally an antioxidant (e.g., BHT); or
(B) potassium peroxymonosulfate (e.g., as MPS triple salt), cross-linked PVP-hydrogen peroxide complex, calcium pyrophosphate, PEG/PPG triblock copolymer (e.g., poloxamer 408), polyvinylpyrrolidone, PEG-PPG random copolymer (e.g., PEG/PPG-116/66), polyethylene glycol (e.g., PEG-600), polyphosphates (e.g., sodium pyrophosphates), anionic surfactant (e.g., sodium lauryl sulfate), a fluoride source (e.g., sodium monofluorophosphate), a thickener (e.g., fumed silica), and one or more sweeteners, flavors, and or colors, and optionally propylene glycol, and optionally an antioxidant (e.g., BHT); or
(C) potassium peroxymonosulfate (e.g., as MPS triple salt), cross-linked PVP-hydrogen peroxide complex, calcium pyrophosphate, PEG/PPG triblock copolymer (e.g., poloxamer 408), PEG-PPG random copolymer (e.g., PEG/PPG-116/66), polyethylene glycol (e.g., PEG-600), propylene glycol, polyphosphates (e.g., sodium pyrophosphates), anionic surfactant (e.g., sodium lauryl sulfate), a fluoride source (e.g., sodium monofluorophosphate), a thickener (e.g., fumed silica), and one or more sweeteners, flavors, and or colors, and optionally an antioxidant (e.g., BHT);

16. A composition according to claim 1, wherein the source of hydrogen peroxide comprises cross-linked PVP complexed with hydrogen peroxide in an amount from 100-25%, by wt., and wherein the cross-linked PVP complexed with hydrogen peroxide provides hydrogen peroxide (e.g., free hydrogen peroxide) in an amount from 0.05-4.5% by wt.).

17. The composition according to claim 1, wherein the composition comprises or consists of:

| Ingredient | Weight % | |
|---|---|---|
| Potassium peroxymonosulfate | 0.1-5% | (e.g., about 1%) |
| Cross-linked Polyvinylpyrrolidone | 5%-20% | (e.g., about 11%) |
| complexed with hydrogen peroxide (e.g., | | |
| 18% hydrogen peroxide relative to the total | | |
| weight of the complex) | | |
| Calcium pyrophosphate | 10-20% | (e.g., about 15%) |
| PEG/PPG triblock copolymer (e.g., | 25-40% | (e.g., about 33%) |
| PLURONIC ® L35) | | |
| Polyvinylpyrrolidone | 1-15% | (e.g., about 5%) |
| PEG-PPG random copolymer (e.g., | 6-15% | (e.g., about 10%) |
| PEG/PPG-116/66) | | |
| Polyethylene glycol (e.g., PEG 600) | 5%-20% | (e.g., about 10%) |
| Propylene Glycol | 0-20% | (e.g., about 7.5%) |
| Polyphosphate (e.g., sodium pyrophosphates) | 2.5-5% | (e.g., about 3.5%) |
| Anionic Surfactant (e.g., sodium lauryl | 1-5% | (e.g., about 2%) |
| sulfate) | | |
| Zwitterionic Surfactant (e.g., | 0.1-1% | (e.g., about 0.3%) |
| cocamidopropyl betaine) | | |
| Fluoride source (e.g., sodium | 0.1 to 2% (e.g., about | |
| monofluorophosphate) | 0.76%) | |
| Antioxidant (e.g., BHT) | 0 to 0.3% (e.g., about | |
| | 0.03%) | |
| Thickener (e.g., fumed silica or sodium | 2.5-5% (e.g., about 3% or | |
| stearate) | 4%) | |
| Sweeteners and flavors, and/or colors | 0.5-5% | |
| Total | Q.S. (ca. 100) | |
| or | | |
| Potassium peroxymonosulfate | 0.1-5% | (e.g., about 1%) |
| Cross-linked Polyvinylpyrrolidone | 1%-10% | (e.g., about 5.5%) |
| complexed with hydrogen peroxide (e.g., | | |
| 18% hydrogen peroxide relative to the total | | |
| weight of the complex) | | |
| Calcium pyrophosphate | 10-40% | (e.g., about 20%) |
| PEG/PPG triblock copolymer (e.g., | 25-40% | (e.g., about 30%) |
| PLURONIC ® L35) | | |
| Propylene Glycol | 0-20% | (e.g., about 7.5%) |
| Polyvinylpyrrolidone | 1-15% | (e.g., about 7.5%) |
| PEG-PPG random copolymer (e.g., | 6-15% | (e.g., about 10%) |
| PEG/PPG-116/66) | | |
| Polyethylene glycol (e.g., PEG 600) | 10-20% | (e.g., about 12%) |
| Polyphosphate (e.g., sodium pyrophosphates) | 2.5-5% | (e.g., about 4%) |
| Anionic Surfactant (e.g., sodium lauryl | 1-5% | (e.g., about 2%) |
| sulfate) | | |
| Fluoride source (e.g., sodium | 0.1 to 2% (e.g., about | |
| monofluorophosphate) | 0.76%) | |
| Antioxidant (e.g., BHT) | 0 to 0.3% (e.g., about | |
| | 0.04%) | |
| Thickener (e.g., fumed silica) | 2.5-5% | (e.g., about 3%) |
| Sweeteners and flavors, and/or colors | 0.5-5% | |
| Total | ca. 100 | |
| or | | |
| Potassium peroxymonosulfate | 0.1-5% | (e.g., about 1%) |
| Cross-linked Polyvinylpyrrolidone | 1%-10% | (e.g., about 5.5%) |
| complexed with hydrogen peroxide (e.g., | | |
| 18% hydrogen peroxide relative to the total | | |
| weight of the complex) | | |
| Calcium pyrophosphate | 5%-40% | (e.g., about 20%) |
| PEG/PPG triblock copolymer (e.g., | 25-40% | (e.g., about 30%) |
| PLURONIC ® L35) | | |
| Polyvinylpyrrolidone | 1-15% | (e.g., about 7.5%) |
| PEG-PPG random copolymer (e.g., | 6-15% | (e.g., about 10%) |
| PEG/PPG-116/66) | | |
| Polyethylene glycol (e.g., PEG 600) | 10-20% | (e.g., about 12%) |
| Propylene Glycol | 0-20% | (e.g., about 7.5%) |
| Polyphosphate (e.g., sodium pyrophosphates) | 2.5-5% | (e.g., about 4%) |
| Anionic Surfactant (e.g., sodium lauryl | 1-5% | (e.g., about 2%) |
| sulfate) | | |
| Fluoride source (e.g., sodium | 0.1 to 2% (e.g., about | |
| monofluorophosphate) | 0.76%) | |
| Antioxidant (e.g., BHT) | 0 to 0.3% (e.g., about | |
| | 0.04%) | |
| Thickener (e.g., fumed silica) | 2.5-5% | (e.g., about 3%) |
| Sweeteners and flavors, and/or colors | 0.5-5% | |
| Total | Q.S. (ca. 100) | |
| or | | |
| Potassium peroxymonosulfate (e.g., as MPS | 0.1-5% | (e.g., about 1%) |
| triple salt) | (e.g., 0.2-10% MPS triple | |
| | salt, e.g., 2.2%) | |
| Cross-linked Polyvinylpyrrolidone | 1%-25% (e.g., about 16- | |
| complexed with hydrogen peroxide (e.g., | 17%) | |
| 18% hydrogen peroxide relative to the total | | |
| weight of the complex) | | |
| Calcium pyrophosphate | 10-40% | (e.g., about 15%) |
| PEG/PPG triblock copolymer (e.g., | 25-40% | (e.g., about 26%) |
| PLURONIC ® L35) | | |
| PEG-PPG random copolymer (e.g., | 6-15% | (e.g., about 10%) |
| PEG/PPG-116/66) | | |
| Polyethylene glycol (e.g., PEG 600) | 10-20% | (e.g., about 10%) |
| Propylene glycol | 5-20% | (e.g., about 7.5%) |
| Polyphosphate (e.g., sodium pyrophosphates) | 2.5-5% | (e.g., about 3%) |
| Anionic Surfactant (e.g., sodium lauryl | 1-5% | (e.g., about 2%) |
| sulfate) | | |
| Fluoride source (e.g., sodium | 0.1 to 2% (e.g., about | |
| monofluorophosphate) | 0.76%) | |
| Antioxidant (e.g., BHT) | 0 to 0.3% (e.g., 0.03%) | |
| Thickener (e.g., fumed silica) | 2.5-5% | (e.g., about 3%) |
| Sweeteners and flavors, and/or colors | 0.5-5% | (e.g., about 3%) |
| | |
|---|---|
| Total | ca. 100 |

18. The composition according to claim 1, wherein the composition is a dentifrice.

19. A method for whitening teeth comprising the steps of (a) applying a composition according to claim 1, to the teeth, and (b) maintaining contact of the composition with the teeth for a sufficient period of time (e.g., 0.1 to 60 minutes, or 0.1 to 30 minutes, or 0.1 to 10 minutes, or 0.1 to 5 minutes, or 0.1 to 2 minutes, or 0.1 to 1 minute) to effect whitening of the teeth contacted by the composition.
